(19) United States
(12) Patent Application Publication
(10) Pub. No.: US 2008/0193938 A1
(43) Pub. Date: Aug. 14, 2008

(54) Materials And Methods Relating To Breast Cancer Classification

(76) Inventors: Yu Kun, Singapore (SG); Patrick Tan, Singapore (SG)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/910,195

(22) PCT Filed: Mar. 30, 2006
(86) PCT No.: PCT/GB2006/001167

§ 371 Date: Sep. 28, 2007

Related U.S. Application Data
(60) Provisional application No. 60/667,063, filed on Apr. 1, 2005.
(60) Provisional application No. 60/671,464, filed on Apr. 15, 2005.

(51) Int. Cl. C12Q 1/68; G01N 33/574; C12M 1/00; C40B 40/08

(52) U.S. Cl. 435/6; 435/7.23; 435/287.2; 506/17

(57) ABSTRACT

The invention provides materials and methods for classifying breast cancer in patients. Particularly there is provided a novel gene expression signature which acts as a predictive signature for response to treatment including hormonal therapies (e.g. tamoxifen) and chemotherapy.

### FIELD OF THE INVENTION

The present invention concerns materials and methods for classifying breast cancers. Particularly, but not exclusively, the invention concerns the classification of breast cancers based on gene expression data. This classification provides important information with regard to patient prognosis (including predicting response to treatment), diagnosis and treatment.

### BACKGROUND OF THE INVENTION

Genome-wide profiling technologies such as DNA microarrays and SAGE are being increasingly used by researchers to characterize the molecular phenotypes of many cancer types. In breast cancer, several groups, including the inventors, have previously used gene expression data to identify various ‘molecular signatures’ of breast tumors and to define clinically-relevant tumor subtypes (1-5). Although much work has been reported on this subject, many of these previous studies have typically employed standard analytical techniques such as hierarchical clustering (HC) and principal components analysis (PCA) to define groups of tumors or genes.

The present inventors have previously shown that a significant proportion of the intrinsic gene expression variation in breast cancer can be attributed to different tumours belonging to distinct ‘molecular subtypes’ (eg ER+ and ER−, and ERBB2+ tumours).

Although these studies have been undoubtedly successful, many of these standard algorithms are also associated with well-known limitations (6, 7). For example, conventional HC algorithms typically cluster genes based on their global behavior across all samples (tumors) in the data set, when in reality certain genes may only show strong regulation in a certain subset of tumors, and weak to minimal regulation in others (1). In addition, standard techniques often do not define relationships between the various molecular signatures, and thus are unable to identify potential interactions between them. Because of these limitations, the inventors believe that a substantial amount of novel biological information still remains deeply embedded within these large-scale data sets, which if unearthed might further our insights into breast cancer biology and lead to improvements in prognosis and treatment.

### SUMMARY OF THE INVENTION

Recently, Barkai and colleagues described a novel analytical approach, signature analysis (SA), which was specifically designed to overcome the problems of conventional clustering (6, 7). When applied to an expression data set, SA identifies independent units termed ‘transcriptional modules’ (TMs), which comprise a group of tightly co-regulated genes in the context of the specific experimental conditions harboring this co-regulation pattern (6). In contrast to most clustering methods where genes are grouped by simultaneously optimizing all clusters, the SA assigns genes to context-dependent and potentially overlapping TMs. SA and its variants have been shown to be superior to conventional clustering algorithms for predicting gene function and defining biological relationships (6, 7).

For the first time, the inventors have applied SA to an in-house set of breast cancer expression profiles. They found that the SA grouped the tumors and genes into distinct modules (termed ‘tumor modules’ (TuMs), to reflect the specific application of SA to cancer), many corresponding to previously reported expression signatures and molecular subtypes for breast cancer. For example, see PCT/GB2004/004195 which is incorporated herein by reference. Besides this proof-of-principle result, the SA surprisingly yielded several novel findings. First, the SA successfully decomposed previously homogenous signatures into independent modules, suggesting that the former might actually consist of multiple related but possibly independent biological programs. Second, the SA revealed a novel apoptosis-related gene signature in Estrogen Receptor (ER+) tumors that was significantly correlated with low histological grade (P<0.001) but independent of ER status. Confidence in the reliability of this signature was obtained by further demonstrating its association with low histological grade in two independent data sets. Third, the SA defined relationships between the tumor modules and uncovered an unexpected positive correlation between ERBB2+ tumors and the immune system, suggesting the presence of substantial cross-talk between these two tissue types.

These results indicate that even after substantial prior analysis, a substantial amount of novel biological information remains embedded within these large-scale data sets, which can be uncovered using the appropriate analytical techniques.

Specifically, the inventors have, for the first time, employed SA to characterize a data set of breast tumor expression profiles. In addition to rediscovering many previously described gene expression signatures in breast cancer, the SA identified a novel gene expression signature (TuM1) that was significantly enriched in genes related to apoptosis and correlated with low histological grade independent of ER status. The TuM1 signature is thus distinct from previously reported expression signatures for low histological grade, which have tended to comprise genes related to ER status, e.g. GATA3 (4).

Importantly, the inventors have further determined that this novel expression signature will function as a predictive signature for response to hormonal therapies in breast cancer. In addition, the over-expression of apoptosis-related genes indicates that such tumors will have enhanced sensitivity to chemotherapy.

Accordingly, at its most general, the present invention provides materials and methods for classifying breast tumors into molecular subtypes and modules using Signature Analysis, particularly Iterative Signature Analysis (ISA); and materials and methods for assigning prognosis and/or treatment regimen to a breast tumor patient based on the SA and ISA of the expression profile of said tumor.

The present invention further provides a method for deriving a set of differentially expressed genes. The invention identifies a set of genes and provides the use of the expression levels of some or all of those genes in a breast tumour sample in assigning a prognosis and/or treatment regimen (e.g. hormonal therapy or chemotherapy) to the patient from whom the sample was derived.

In a first aspect, the present invention provides a method for determining the prognosis of a patient with breast cancer, the method comprising assigning a prognosis to the patient based on the expression levels in a breast tumour of said patient of a set of genes (hereafter referred to as the “prognostic set”), wherein the prognostic set includes a plurality of genes from TuM1 as shown in Table 2.

The invention further provides the use of the prognostic set in determining the prognosis and/or treatment regimen of a patient with breast cancer. Preferably, the invention provides the use of an expression profile in determining the prognosis and/or treatment of a patient with a breast tumour, the expression profile representing the expression levels in the tumour of the genes of the prognostic set.

“Prognosis” is intended in its most general sense, and may be quantitative or qualitative. It may be expressed in general terms, such as a “good” or “bad” prognosis, and/or in terms of likely clinical outcomes, such as duration of disease free survival (DFS), likelihood of survival for a defined period of time, and/or probability of distant metastasis within a defined period of time. Quantitative measures of prognosis will generally be probabilistic. Additionally or alternatively, and especially for communicating the prognosis to or between medical practitioners, the prognosis may be expressed in terms of another indicator of prognosis, such as the Nottingham Prognostic Index (NPI) scale.

In general, a patient with a ‘good prognosis’ tumour would probably be treated with a conventional treatment regimen. A patient with a ‘poor prognosis’ tumour might be treated with an alternative or more aggressive regimen. The ‘poor prognosis’ patient would usually not have to wait for the conventional treatment regimen to fail before moving onto the more aggressive one. Furthermore, having an understanding of the likely clinical course of the disease allows a patient to prepare a realistic plan for future, which is an important social aspect of cancer treatment.

As mentioned above, the inventors have determined that the TuM1 expression signature predicts that the patient will respond well to hormonal treatment and to chemotherapy. Consequently, the prognostic set mentioned above may be used to predict the response to treatment, in particular hormonal treatment (e.g. tamoxifen or indeed any selective modulators of estrogen receptors) or chemotherapy.

For the avoidance of doubt, the term “determining” need not imply absolute certainty in prognosis. Rather, the expression levels of the prognostic set in a tumour will generally be indicative of the likely prognosis of the patient.

The expression levels will generally be represented numerically. The expression profile therefore will generally include a set of numbers, each number representing the expression level of a gene of the prognostic set.

A method in accordance with the first aspect of the invention may comprise the steps of:
providing an expression profile that represents the expression levels in the tumour of the genes of the prognostic set, and
assigning a prognosis and/or treatment regimen to the patient based on the expression profile.

The providing step may include extracting information on the expression levels of the genes of the prognostic set from a pre-existing data set, which may also include other expression levels (e.g. data representing expression levels of other genes in the tumour). Alternatively, it may include determining the expression levels experimentally.

The determining step may include the steps of:
(a) obtaining a breast tumour sample from the patient;
(b) measuring the expression levels in the sample of the genes of the prognostic set.

Measurement of the expression level of a gene, and in particular its representation in the expression profile, may be in absolute terms, or relative to some other factor such as, but not limited to, the expression of another gene, or a mean, median or mode of the expression level of a group of genes (preferably genes outside the prognostic set, but possibly including genes of the prognostic set) in the sample or across a group of samples. For example, expression of a gene may be measured or represented as a multiple or fraction of the average expression of a plurality of genes in the sample. Preferably, the expression is represented in the expression profile as positive or negative to indicate an increase or decrease in expression relative to the average value.

In a non-preferred embodiment, expression profile information in the form of a set of numerical values is converted into a ranked list of genes of the prognostic set, wherein the genes are ranked in order of expression level, after which the rank order of the individual genes is used as a parameter in the analysis (instead of the expression value of the gene).

Preferably, step (b) comprises contacting said expression products obtained from the sample with a plurality of binding members capable of binding to expression products that are indicative of the expression of genes of the prognostic set, wherein such binding may be measured.

Generally, the binding members are capable of not only detecting the presence of an expression product but its relative abundance (i.e. the amount of product available). The expression profile can be determined using binding members capable of binding to the expression products of the prognostic set, e.g. mRNA, corresponding cDNA or cRNA or expressed polypeptide. By labelling either the expression product or the binding member it is possible to identify the relative quantities or proportions of the expression products and determine the expression profile of the prognostic set. The binding members may be complementary nucleic acid sequences or specific antibodies.

The step of assigning a prognosis may be carried out by comparing the expression profile under test with other, previously obtained, profiles that are associated with known prognoses and/or with a previously determined “standard” profile (or profiles) which is (or are) characteristic of a particular prognosis (or prognoses). A standard profile for a particular prognosis may be generated from expression profiles from a plurality of tumours of that prognosis.

The comparison will generally be performed by, or with the aid of, a computer.

Preferably the expression profile is compared with known or standard profiles (preferably standard profiles) of differing known prognoses. The prognosis to be assigned to the patient is that of the known or standard profile which the expression profile under test most closely resembles. The standard profiles used for comparison may also be used to assign a treatment regimen.

Preferably the comparison is with known or standard profiles (preferably standard profiles) that are categorised into two different prognoses, e.g. “good” and “bad”, or high and low (preferably with a cut-off between 3.8 and 4.6). The known or standard profiles will have been generated from samples of known prognosis, which may be determined in any convenient way—either by actual clinical outcome for the patient following the removal of the sample (i.e. response to treatment), or by other prognostic techniques, e.g. histopathological techniques, e.g. using the NPI scale.

The known or standard profiles may also have been generated from samples which have undergone a particular treatment regimen, e.g. hormonal treatment and/or chemotherapy, and where the clinical outcome is known.

Advantageously, the use of a gene expression profile to assign a prognosis and/or a treatment regimen may reduce or may even eliminate the subjective nature of the clinical procedures used to assign a prognosis to a tumour sample. As the method requires assessment of expression products at the molecular level, preferably quantitatively, the method provides a more objective, and therefore potentially more reliable, way to assign a prognosis. The prognostic set is capable of separating breast tumour samples into discrete modules, and therefore reducing, or even eliminating, the subjective analysis of clinical prognostic assignment. Furthermore, a confidence can be assigned to the prediction, so that an informed choice regarding treatment of the patient can be made, depending on the “strength” of the prognosis.

The expression profile of the prognostic set may differ slightly between independent samples of similar prognosis. However, the inventors have realised that the expression profile of the particular genes that make up the prognostic set when used in combination provide a pattern of expression (expression profile) in a tumour sample, which pattern is characteristic of the tumour's prognosis.

The prognostic set of the invention (TuM1 (Table 2 and Table 2a)) is a subgroup of ER+ tumors. The TuM1 expression signature appears to be a specific molecular feature of ER+ low histological grade tumors independent of ER status.

The expression profile obtained from a patient using the prognostic set will provide valuable information not only for prognosis but for a possible treatment regimen.

The treatment may be chemotherapy and/or hormonal treatment, e.g. tamoxifen or other selective modulators of estrogen receptors.

The methods of the invention may include comparing the expression levels of the prognostic set in the breast tumour sample before and after treatment to detect a change in the expression profile indicative of an improved prognosis or worsened prognosis.

The expression profile represents the expression levels of a group of genes in the tumour. The genes of each expression profile need not be identical but there should be sufficient overlap between the genes of each expression profile to allow comparison and grouping of the expression profiles.

The binding member may be labelled for detection purposes using standard procedures known in the art. Alternatively, the expression products may be labelled following isolation from the sample under test. A preferred means of detection is using a fluorescent label which can be detected by a light meter. Alternative means of detection include electrical signaling. For example, the Motorola (Pasadena, Calif. ) e-sensor system has two probes, a “capture probe” which is freely floating, and a “signaling probe” which is attached to a solid surface which doubles as an electrode surface. Both probes function as binding members to the expression product. When binding occurs, both probes are brought into close proximity with each other resulting in the creation of an electrical signal which can be detected.

There are, however, a number of newer technologies that have recently emerged that utilize ‘label-free’ techniques for quantitation, for example those produced by Xagros (Mountain View, Calif.). The primers and/or the amplified nucleic acid may be devoid of any label. Quantitation may be assessed by measuring the change in electrical resistance as a result of two primers docking onto a target expressed product, and subsequent extension by polymerase.

As discussed above, the binding members may be oligonucleotide primers for use in a PCR (e.g. multi-plexed PCR) to amplify specifically the number of expressed products of the genetic identifiers. The products would then be analysed on a gel. However, preferably, the binding member is a single nucleic acid probe or antibody fixed to a solid support. The expression products may then be passed over the solid support, thereby bringing them into contact with the binding member. The solid support may be a glass surface, e.g. a microscope slide; beads (Lynx); or fibre-optics. In the case of beads, each binding member may be fixed to an individual bead and they are then contacted with the expression products in solution.

Various methods exist in the art for determining expression profiles for particular gene sets and these can be applied to the present invention. For example, bead-based approaches (Lynx) or molecular bar-codes (Surromed) are known techniques. In these cases, each binding member is attached to a bead or “bar-code” that is individually readable and free-floating to ease contact with the expression products. The binding of the binding members to the expression products (targets) is achieved in solution, after which the tagged beads or bar-codes are passed through a device (e.g. a flow-cytometer) and read.

A further known method of determining expression profiles is instrumentation developed by Illumina (San Diego, Calif.), namely, fibre-optics. In this case, each binding member is attached to a specific “address” at the end of a fibre-optic cable. Binding of the expression product to the binding member may induce a fluorescent change which is readable by a device at the other end of the fibre-optic cable.

In a second aspect, the present invention provides apparatus, preferably a microarray, for assigning a prognosis and/or treatment regimen to a breast tumour sample, which apparatus comprises a solid support to which are attached a plurality of binding members, each binding member being capable of specifically binding to an expression product of a gene of the prognostic set. Preferably the binding members attached to the solid support are capable of specifically and independently binding to expression products of at least 5 genes, more preferably, at least 10 genes or at least 15 genes, and most preferably at least 20 or 30 genes identified in Table 2. The binding members attached to the solid support may be capable of specifically binding to expression products of 20 to 30 genes identified in Table 2.

In one embodiment, binding members being capable of specifically and independently binding to expression products of all genes identified in Table 2 are attached to the solid support. The support may have attached thereto only binding members that are capable of specifically and independently binding to expression products of the genes identified in Table 2, or a prognostic set therefrom.

Preferably the binding members are nucleic acid sequences and the apparatus is a nucleic acid microarray.

The genes of Table 2 are listed with their Unigene accession of the Unigene database. The sequence of each gene can therefore be retrieved from the Unigene database at the National Institute of Health (NIH):
(http://www.ncbi.nlm.nih.gov/entrez/guery.fcgi?db=unigene).

Table 2a lists the genes of Table 2 in order of significance. Thus, for all aspects of the present invention it is preferable that the set of genes selected from Table 2 comprises at least the first 5 genes listed in Table 2a, more preferably, at least the first 6, 7, 8, 10, 12, 15, 17, 20, 25, 30 genes listed in Table 2a.

Thus, in a preferred embodiment of the present invention, the set of genes comprises at least 10 genes selected from Table 2 wherein at least 5 of those genes are the first five genes listed in Table 2a.

The set of genes may comprises at least 15, 20, 25 or 30 genes selected from Table 2 where at least 5, 10, 15, 20 or 25 of those genes are the first 5, 10, 15, 20 or 25 genes listed in Table 2a.

Furthermore, for all of the genes, Affymetrix (Santa Clara, Calif.) (www.affymetrix.com) provide examples of probe sets, including the sequences of the probes, (i.e. binding members in the form of oligonucleotide sequences) that are capable of detecting expression of the gene when used on a solid support.

Typically, high density nucleic acid sequences, usually cDNA or oligonucleotides, are fixed onto very small, discrete areas or spots of a solid support. The solid support is often a microscopic glass side or a membrane filter, coated with a substrate (i.e. a “chip”). The nucleic acid sequences are delivered (or printed), usually by a robotic system, onto the coated solid support and then immobilized or fixed to the support.

In a preferred embodiment, the expression products derived from the sample are labelled, typically using a fluorescent label, and then contacted with the immobilized nucleic acid sequences. Following hybridization, the fluorescent markers are detected using a detector, such as a high resolution laser scanner. In an alternative method, the expression products could be tagged with a non-fluorescent label, e.g. biotin. After hybridisation, the microarray could then be ‘stained’ with a fluorescent dye that binds/bonds to the first non-fluorescent label (e.g. fluorescently labelled strepavidin, which binds to biotin). The expression products may, however, be label-free, as discussed above.

A binding profile indicating a pattern of gene expression (expression pattern or profile) is obtained by analysing the signal emitted from each discrete spot with digital imaging software. The pattern of gene expression of the experimental sample may then be compared with that of a standard profile (i.e. an expression profile from a tissue sample with, for example, a known good or bad prognosis, or a known NPI value or known range of NPI values) for differential analysis.

The standard may be derived from one or more expression profiles previously judged to be characteristic of a particular prognosis e.g. ‘poor’ or ‘good’ prognosis and/or of a particular NPI range such as high and/or low NPI and/or characteristic of one or more NPI value(s) or one or more range(s) of values. The standard may be derived from one or more expression profiles previously judged to be characteristic of a particular NPI value or range of values (or other defined value on a prognostic scale). The standard may include an expression profile characteristic of a normal sample. These/This standard expression profile(s) may be retrievably stored on a data carrier as part of a database.

Most microarrays utilize either one or two fluorophores. For two-colour arrays, the most commonly used fluorophores are Cy3 (green channel excitation) and Cy5 (red channel excitation). The object of the microarray image analysis is to extract hybridization signals from each expression product. For one-colour arrays, signals are measured as absolute intensities for a given target (essentially for arrays hybridized to a single sample). For two-colour arrays, signals are measured as ratios of two expression products, (e.g. sample and control (controls are otherwise known as a ‘reference’)) with different fluorescent labels.

The apparatus in accordance with the present invention preferably comprises a plurality of discrete spots, each spot containing one or more oligonucleotides and each spot representing a different binding member for an expression product of a gene selected from Table 2. In one embodiment, the microarray will contain spots for each of the genes provided in Table 2. Each spot will comprise a plurality of identical oligonucleotides each capable of binding to an expression product, e.g. mRNA or cDNA, of the gene of Table 2 it is representing. Each gene is preferably represented by a plurality of different oligonucleotides.

In a third aspect of the present invention, there is provided a kit for assigning a prognosis and/or treatment regimen to a patient with breast cancer, said kit comprising a plurality of binding members capable of specifically binding to expression products of genes of the prognostic set, and a detection reagent. The kit may include a data analysis tool, preferably in the form of a computer program. The data analysis tool preferably comprises an algorithm adapted to discriminate between the expression profiles of tumours with differing prognoses.

In one embodiment, the kit includes apparatus of the second aspect of the invention.

Preferably, the one or more binding members (antibody binding domains or nucleic acid sequences e.g. oligonucleotides) in the kit are fixed to one or more solid supports e.g. a single support for microarray or fibre-optic assays, or multiple supports such as beads. The detection means is preferably a label (radioactive or dye, e.g. fluorescent) for labelling the expression products of the sample under test. The kit may also comprise reagents for detecting and analysing the binding profile of the expression products under test.

Alternatively, the binding members may be nucleotide primers capable of binding to the expression products of genes identified in Table 2 such that they can be amplified in a PCR. The primers may further comprise detection means, i.e. labels that can be used to identify the amplified sequences and their abundance relative to other amplified sequences.

The breast tumour sample may be obtained as excisional breast biopsies or fine-needle aspirates.

In a fourth aspect, there is provided a method of producing a nucleic acid expression profile for a breast tumour sample comprising the steps of
(a) isolating expression products from said breast tumour sample;
(b) identifying the expression levels of the prognostic set of genes; and
(c) producing from the expression levels an expression profile for said breast tumour sample.

The expression profile may be added to a gene expression profile database. The method may further comprise the step of comparing the expression profile with a second expression profile (or a plurality of second expression profiles). The second expression profile (or profiles) may be produced from a second breast tumour sample (or samples) using substantially the same prognostic set, wherein a prognosis has been assigned to, or determined for, the second sample (or samples). The second expression profile (or profiles) may be a standard profile (or profiles) characteristic of a particular prognosis, for example a ‘good’ prognosis or a ‘poor’ prognosis, or a high NPI or a low NPI, or at least one particular NPI value or at least one range of NPI values. Alternatively, or as well, the standard profile (or profiles) may indicate a particular treatment regimen.

Preferably the prognosis is in the form of a prognostic measure, preferably a clinically accepted prognostic classification system, such as the NPI. Again, the prognosis may be predicted from gene expression data, derived from clinical techniques, such as histopathological techniques, or assigned retrospectively to the second expression profile based on the disease outcome of the patient(s) that contributed sample(s) from which the second profile was derived.

With knowledge of the prognostic set, it is possible to devise many methods for determining the expression pattern or profile of the genes in a particular test sample. For example, the expressed nucleic acid (RNA, mRNA) can be isolated from the sample using standard molecular biological techniques. The expressed nucleic acid sequences corresponding to the gene members of the genetic identifiers given in Table 2 can then be amplified using nucleic acid primers specific for the expressed sequences in a PCR, If the isolated expressed nucleic acid is mRNA, this can be converted into cDNA for the PCR reaction using standard methods.

The primers may conveniently introduce a label into the amplified nucleic acid so that it may be identified. Ideally, the label is able to indicate the relative quantity or proportion of nucleic acid sequences present after the amplification event, reflecting the relative quantity or proportion present in the original test sample. For example, if the label is fluorescent or radioactive, the intensity of the signal will indicate the relative quantity/proportion or even the absolute quantity, of the expressed sequences. The relative quantities or proportions of the expression products of each of the genetic identifiers will establish a particular expression profile for the test sample.

The classification of the expression profile is more reliable the greater number of gene expression levels tested. The known microarray and genechip technologies allow large numbers of binding members to be utilized. Therefore, the more preferred method would be to use binding members representing all of the genes in Table 2. However, the skilled person will appreciate that a proportion of these genes may be omitted and the method still carried out in a reliable and statistically accurate fashion.

The prognostic set in any aspect of the invention may comprise, or consist of, all, or substantially all, of the genes from Table 2. The prognostic set of genes may vary in content and number, independently, between aspects of the invention.

The prognostic set may include at least 5, 10, 20, 30 or all of the genes of Table 2.

The provision of the prognostic set allows diagnostic tools, e.g. nucleic acid microarrays to be custom made and used to predict, diagnose or subtype tumours. Further, such diagnostic tools may be used in conjunction with a computer which is programmed to determine the expression profile obtained using the diagnostic tool (e.g. microarray) and compare it, as discussed above, to a “standard” expression profile or a database of expression profiles of ‘known’ prognosis. In doing so, the computer not only provides the user with information which may be used diagnose the presence or type of a tumour in a patient, but at the same time, the computer obtains a further expression profile by which to determine the ‘standard’ expression profile and so can update its own database.

Thus, the invention allows, for the first time, specialized chips (microarrays) to be made containing probes corresponding to the prognostic set. The exact physical structure of the array may vary and range from oligonucleotide probes attached to a 2-dimensional solid substrate to free-floating probes which have been individually “tagged” with a unique label, e.g. “bar code”.

Querying a database of expression profiles with known prognosis can be done in a direct or indirect manner. The “direct” manner is where the patient's expression profile is directly compared to other individual expression profiles in the database to determine which profile (and hence which prognosis and/or treatment regimen) delivers the best match.

Aspects and embodiments of the present invention will now be illustrated, by way of example, with reference to the following figures. Further aspects and embodiments will be apparent to those skilled in the art. All documents mentioned in this text are incorporated by reference.

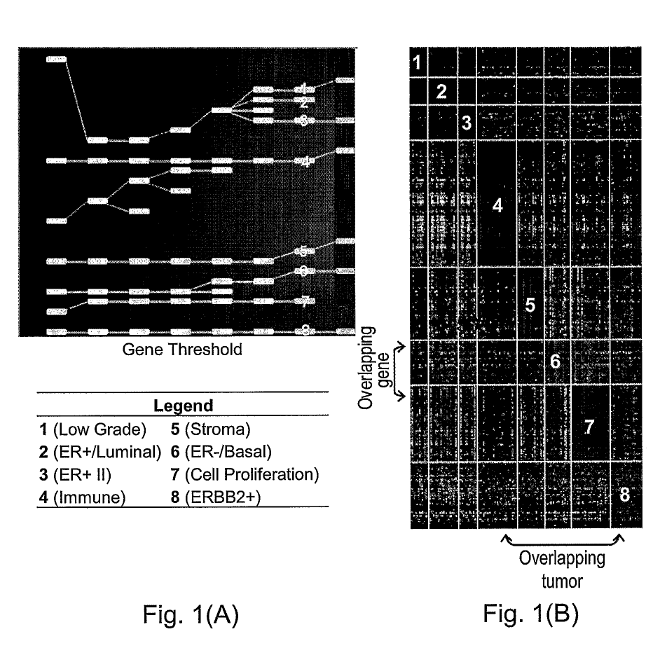

FIG. 1. Tumor Modules of Breast Cancer. A) The module tree of the tumor modules (TuMs) identified by the ISA at different resolution levels. Each node (solid blue rectangle) represents a transcriptional module. Branches represent TuMs that originate from same roots over a range of thresholds. B) Global view of gene expression patterns within Tumor Modules. Each row represents one gene and each column represents one tumor. Eight diagonal blocks (separated by yellow grid) represent eight modules (under gene threshold 3.0) from FIG. 1A). The legend of eight modules is listed. The off-diagonal blocks reveals how genes in one module function in other modules. The red arrows show examples of genes and tumors that can be shared between different modules.

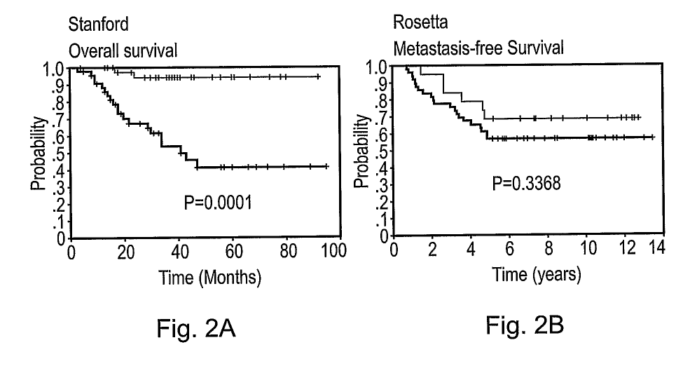

FIG. 2. Kaplan-Meier analysis of disease outcome in two independent patient groups. A) Overall survival for 82 ER+ patients from Stanford data set. B) Metastasis-free survival for 71 ER+ patients from the Rosetta data set. The green line indicates patients with ER positive tumors highly expressing TuM1 genes; while the pink one depicts patients with all other ER+ tumors.

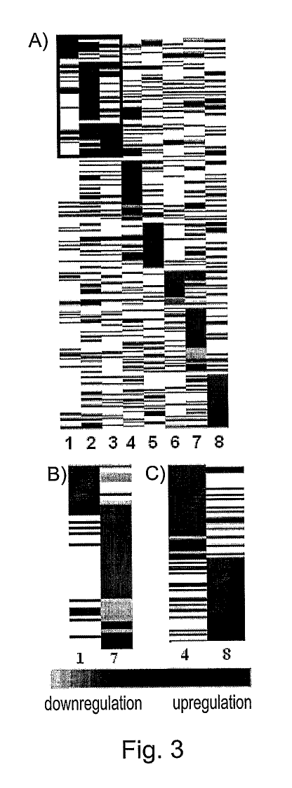

FIG. 3. Correlations Between the Tumor Signatures of Different Modules. Each row represents a tumor, where the color of the line varies according to the score assigned to that tumor (color bar). A) Global visualization of co-regulation among the eight TuMs. The diagonal boxes are modules with corresponding tumors. The lines in off-diagonal boxes show the tumors shared by other modules. Comparing the color of one line in diagonal to off-diagonal boxes reveals the extent of correlation between two modules. TuM1, TuM2 and TuM3 are highlighted with a blue rectangle. B) Correlations between TuM1 (low grade) and TuM7 (cell proliferation) tumors; and C) TuM4 (immune response) and TuM8 (ERBB2+) tumors.

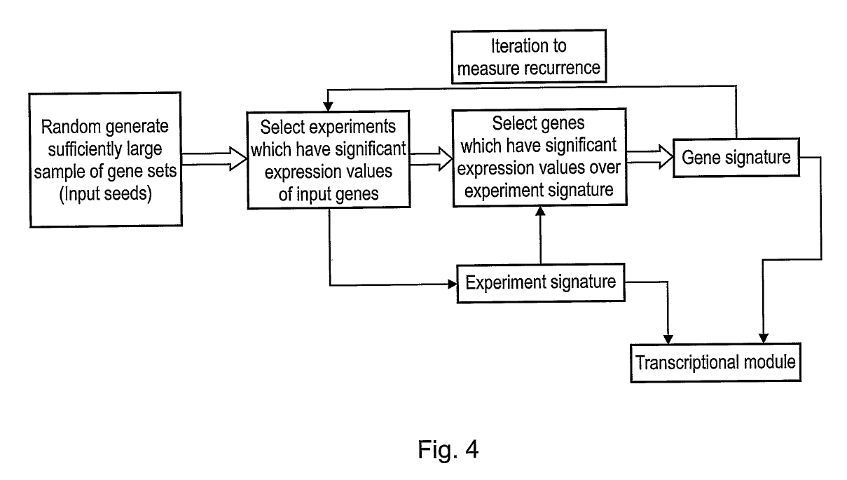

FIG. 4 shows the workflow of the Iterative Signature Algorithm (ISA)

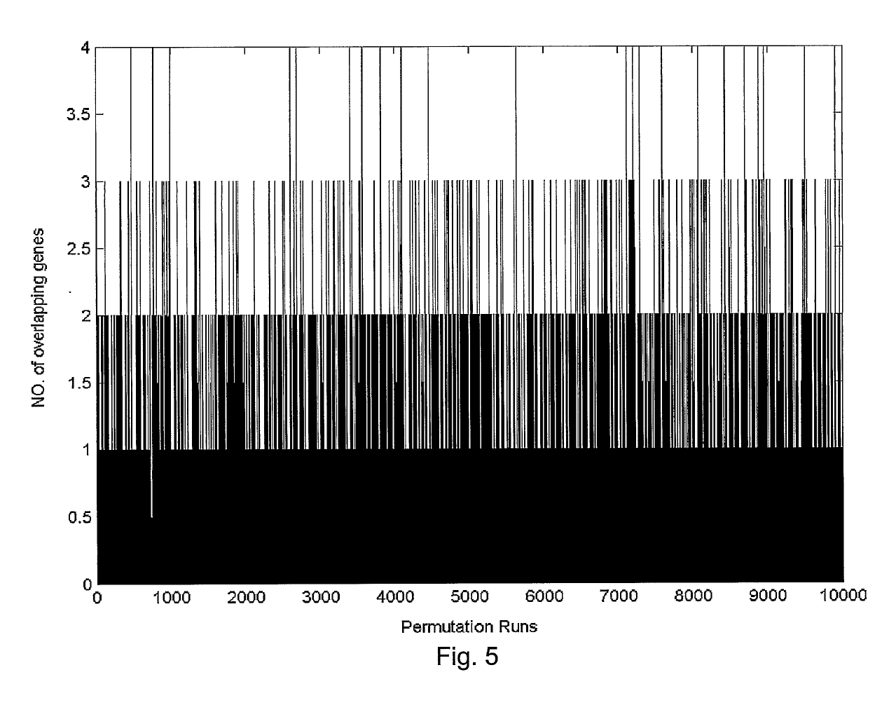

FIG. 5 shows the genes overlapping between TuM7 and NPI-ES.

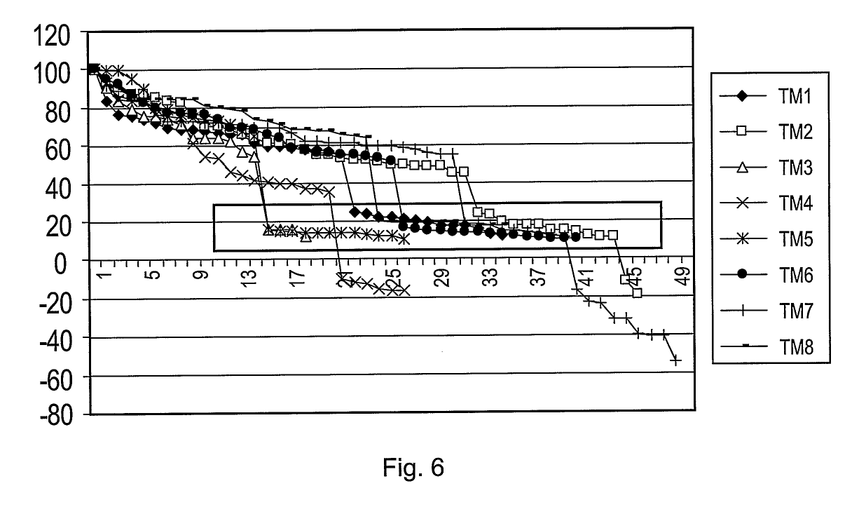

FIG. 6. The tumor scores of the transcriptional modules. Tumors are sorted by their tumor score. Y-axis is the tumor score. X-axis is the index of the tumor, which varies in different modules.

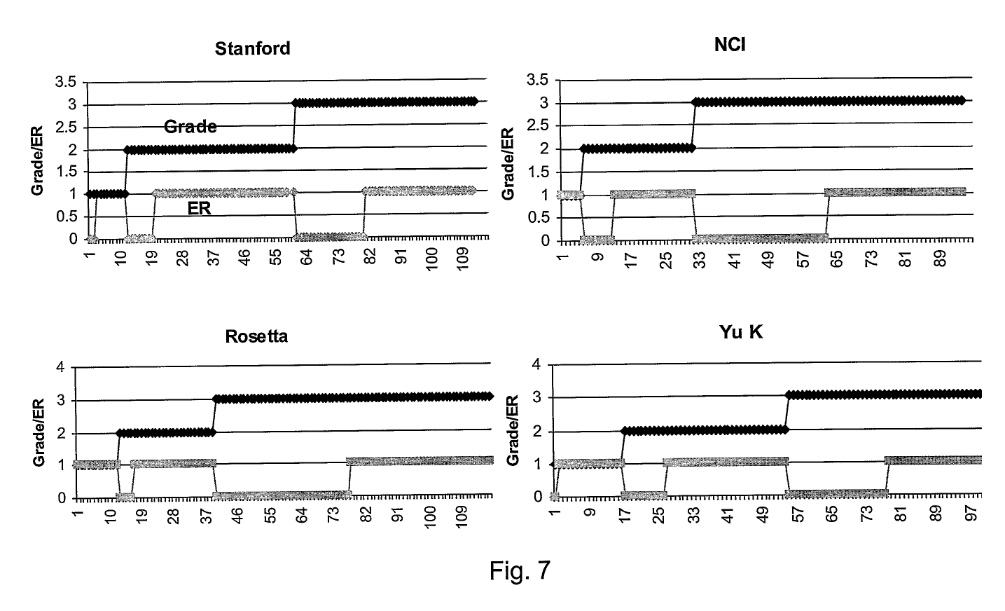

FIG. 7. The distribution of grade and ER status in various breast cancer data sets. The dark line is grade; the light line is ER status. Y-axis showed the grade (1-3). ER-positive was assigned as 1; while ER-negative was 0. The samples were sorted by grade, and by ER subsequently.

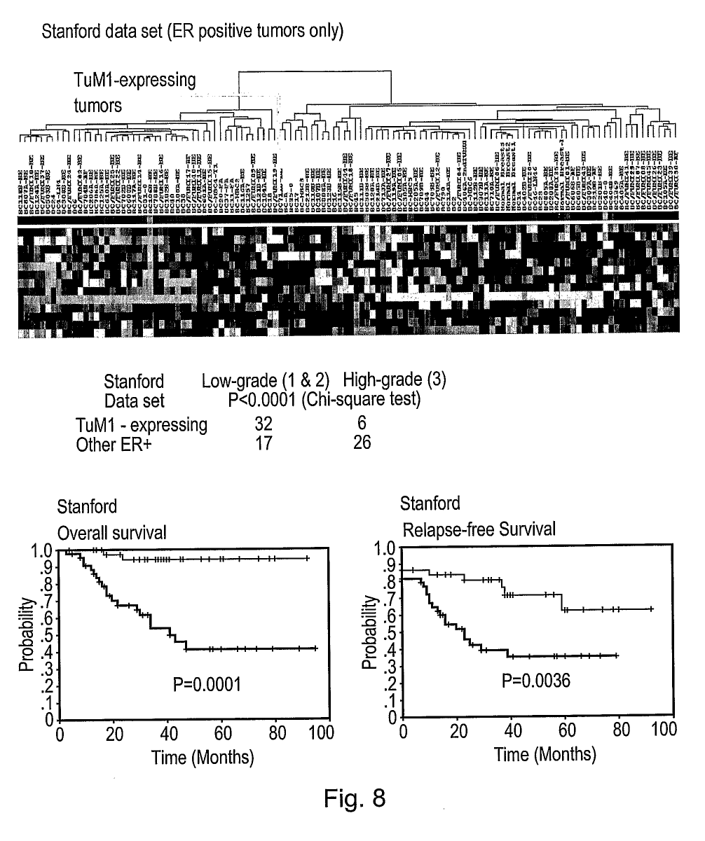

FIG. 8. shows Stanford data set (ER positive tumors only)

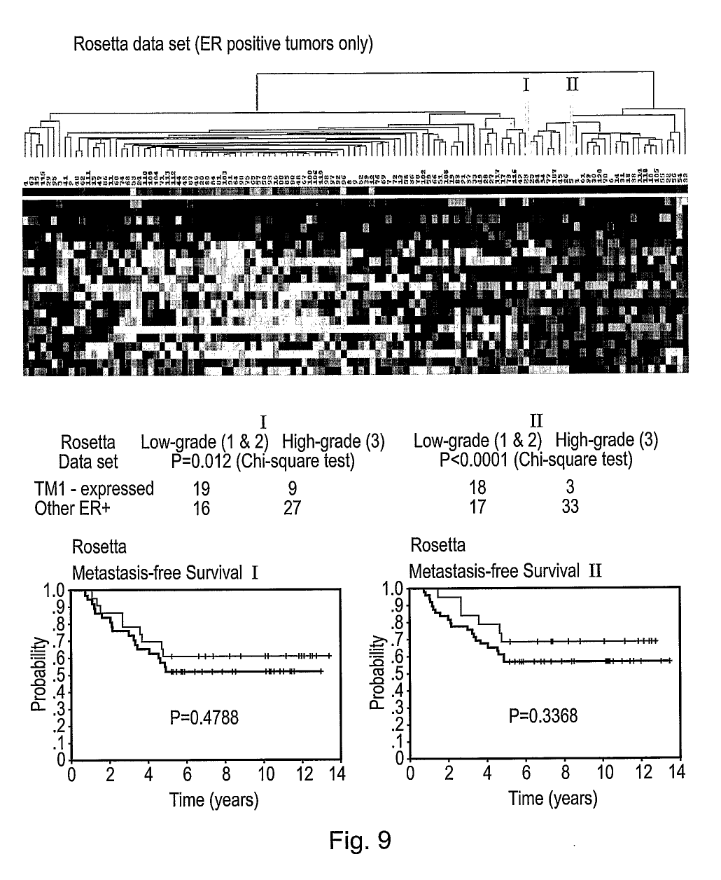

FIG. 9. Rosetta data set (ER positive tumors only)

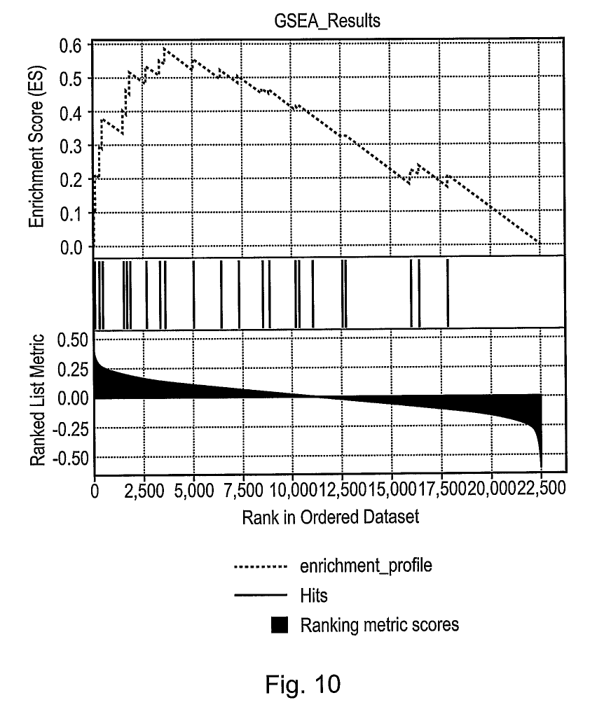

FIG. 10. Gene set enrichment analysis. Genes are ranked by the signal-to-noise (S2N) ratio on control vs. treated cell line. The higher S2N ratio (rank), the lower expression values in treated cell line compared to control.

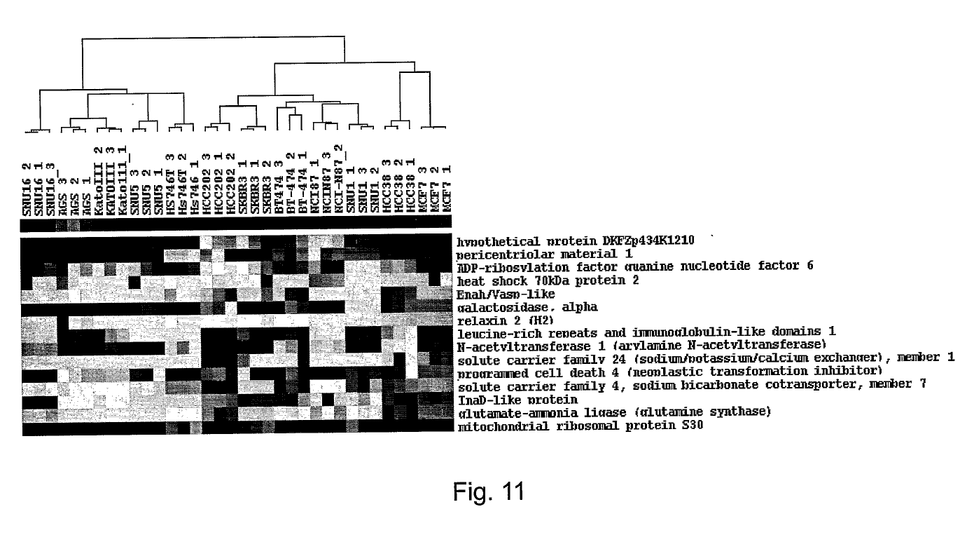

FIG. 11. Hierarchical clustering of various cell lines on the basis of expression profiling of TuM1 genes. Average-linkage hierarchical clustering employing a Pearson correlation metric was used in this analysis. The overexpression of TuM1 genes in MCF7 is highlighted in a yellow rectangle.

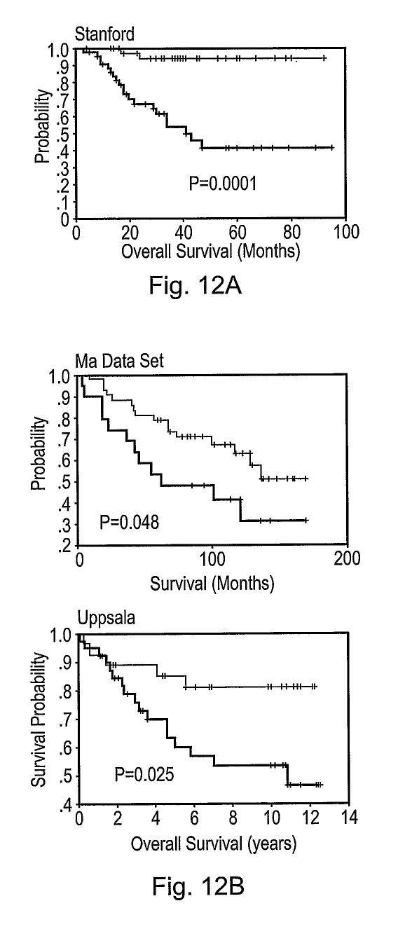

FIG. 12. Multivariate analysis of risk factors for death (Uppsala and Stanford) or metastasis (Ma) as the first event—see also Table 6.

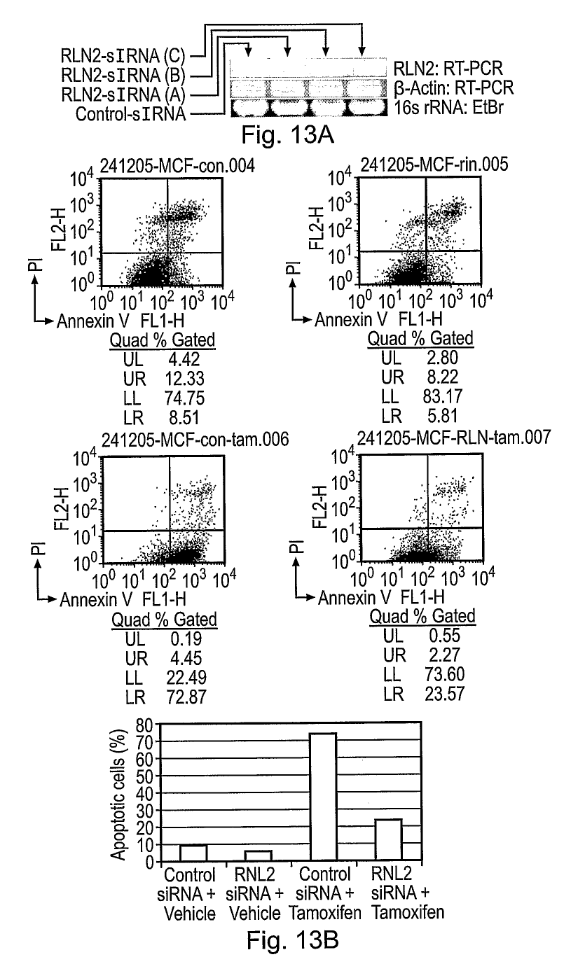

FIG. 13A. RLN2 gene silencing in MCF-7 cells MCF-7 cells were transfected with RLN2 specific siRNAs representing 3 different regions of the gene and the RLN2 mRNA quantity was analyzed at 72 hrs. The efficient siRNA (C) in combination with siRNA (B) was used to knockdown RLN2 in Tamoxifen responsiveness assay.

FIG. 13B. Flow cytometric analysis of Tamoxifen sensitivity in RLN2 silenced MCF-7 cells: RLN2 silenced and control cells were treated with 1 μm Tamoxifen or equivalent quantity of vehicle for 48 hrs and subsequently, the treatment was withdrawn. After 72 hrs, Annexin-V staining positive cells were scored in Flow cytometry, which is a measure for tamoxifen induced apoptosis.

### MATERIALS AND METHODS

Breast Tissues and Clinical Information

A total of 96 beast invasive carcinomas were obtained from the National Cancer Centre of Singapore (NCC) Tissue Repository, after appropriate approvals from the NCC Repository and Ethics Committees. Profiled samples contained at least 50% tumor content. Detailed descriptions of sample collection, archiving, and histological assessment of tumors, including techniques and parameters, have been previously reported (5).

Sample Preparation and Microarray Hybridization

RNA was extracted from tissues using Trizol (Invitrogen, Carlsbad, Calif.) reagent and processed for Affymetrix Genechip (Affymetrix Inc., Santa Clara, Calif.) hybridizations using U133A Genechips according to the manufacturer's instructions.

Data Processing

Raw Genechip scans were quality controlled using GeneData™ Refiner (Genedata, Basel, Switzerland) and deposited into a central data storage facility. The expression data was pre-processed by removing genes whose expression was absent throughout all samples (ie ‘A’ calls), subjecting the remaining genes (9116 probes) to a log2 transformation, and normalization by median-centering of samples.

Signature Algorithm (SA) and Iterative Signature Algorithm (ISA)

A detailed description of the SA methodology is provided in ref 6 which is incorporated herein by reference. Briefly, the SA operates as follows: 1) A selected set of ‘input genes’ are fed to the SA algorithm; 2) The SA selects those tumors in which the average expression of the input genes is above a pre-defined threshold; 3) The global profiles of these selected tumors are then examined to select other genes whose average expression is above a gene threshold. The output of SA is a ‘tumor module’ (TuM), comprising a set of genes that display expression levels above a particular gene threshold within a specific group of tumors. The inventors utilize an extension of SA, the iterative signature algorithm (ISA), which utilizes a large number of random gene sets as the initial input genes and subsequently refines the TuMs through multiple iterative rounds of SA (7). As the inputted genes are random, ISA does not require prior knowledge and hence constitutes an entirely unsupervised analytical approach. Based upon previous reports, a gene threshold of 3.0 was selected as an optimal threshold for further in-depth analysis (6). The lists of genes within each TuM are contained in the Supplementary Information. Correlations between tumor modules were calculated as described in (6).

The SA software is available at: http://barkai-serv.weizmann.ac.il/GroupPage/software.htm.

Associations Between TuMs and Clinical Data

Chi-square tests were used to calculate the association between each TuM and the following clinical parameters: patient age, lymph node (LN) status, estrogen receptor (ER) status, progesterone receptor (PR) status, tumor size, histological grade, and lymphovascular invasion (LVI). The significance of each association was also confirmed by hypergeometric probability density function analysis.

Techniques

Human breast tissues were obtained from the NCC Tissue Repository, after appropriate approvals from the NCC Repository and Ethics Committees. Samples were grossly dissected in the operating theater immediately after surgical excision, and flash-frozen in liquid N2. Samples had not been treated with pre-operative chemotherapy. For histological assessment of tumors and axillary lymph nodes, formalin-fixed, paraffin-embedded tumor tissue was used to determine tumor subtype (WHO classification), histologic grade, and lymphovascular invasion. Tumor size, based only on the invasive component, was assessed macroscopically and confirmed microscopically. For small tumors, the size was measured on this histologic section. ER status was determined by immunohistochemistry, with a positive result being >10% of carcinoma cells showing nuclear reactivity of at least +2 intensity. For ERBB2 immunohistochemistry, the Dako classification system was used with scores of 0 and 1+ considered negative while 2+ and 3+ were positive. An indeterminate conclusion was made when benign breast epithelium was immunoreactive. Profiled samples contained at least 50% tumor content.

ISA Work Scheme

The Iterative signature algorithm (ISA) is an extension of the basic signature algorithm that can be used to globally decompose gene expression data. In general, the ISA is a self-feed system and applied as follows: 1) generate a (sufficiently) large sample of input seeds; 2) identify the robust modules (similar to SA) corresponding to each seed through multiple iterations. FIG. 4 depicts the ISA schema. A detailed technical report of ISA can be found in Bergmann et al., March 2003; 67(3 Pt 1):031902. The parameters used are shown as follows. Definitions of each parameter can be found in: http://barkai-serv.weizmann.ac.il/GroupPage/software.htm.

Parameter settings of ISA

| | Gene | | | |
|---|---|---|---|---|
| Condition | Threshold | | | |
| Threshold | range | minRecurrence | minNoGenes | randomSizes |
| 3 | [1.8, 4] | 2 | 10 | [5, 10:1:20] |

Correlation of Grade to ER Status

To study the relations between grade and ER status, the inventors surveyed four breast cancer data sets: 1) Standford data set (ref. 3); 2) NCI data set (ref. 4); 3) Rosetta data set (ref. 10); and 4) their in-house data set. FIG. 7 showed the grade and ER status for each breast tumor. The trend that the ER negative tumors are high-grade is obvious.

Cell Culture and Tamoxifen Treatment

MCF-7 breast cancer cells were obtained from American Type Culture Collection center (Manassas, Va.), and cells were cultured in Dulbecco's modified Eagle medium (DMEM) (Gibco, Grand Island, N.Y.) supplemented with 10% fetal bovine serum (FBS), 100 U/mL penicillin, 100 U/mL streptomycin, and 2 mM L-glutamine. Before tamoxifen treatment, cells were washed three times in PBS and maintained in phenol red free DMEM with 5% Dextran charcoal-stripped FBS (HyClone Laboratories, Pittsburgh, Pa.) for 24 hrs. Subsequently cells were treated with 10 μM tamoxifen (Sigma) and harvested at 48 hrs. Control sister cultures were treated with an equivalent volume of the vehicle (0.1% ethanol).

Gene Set Enrichment Analysis

GSEA was used to ask if expression of the tumor module genes might be affected by tamoxifen treatment. Four control samples and two post-treatment samples (See Materials and Methods) were used for GSEA analysis. Three modules (TuM4, 5 and 6) were filtered out due to insufficient number of genes (<10) expressed in MCF7 cell lines. TuM1 is the sole module showed a significant correlation with control samples (ie, downregulated in treated MCF7 cell line; see table and FIG. 10).

| | | | | | | |
|---|---|---|---|---|---|---|
| | | | | NOM | | FWER |
| NAME | SIZE | ES | NES | p-val | FDR q-val | p-val |

downregulated in treated MCF7 cells

| | | | | | | |
|---|---|---|---|---|---|---|
| TuM1 | 16 | 0.616471 | 1.6929 | 0 | 0.05 | 0 |
| TuM2 | 16 | 0.727534 | 1.426171 | 0 | 0.19 | 0.15 |
| TUM7 | 33 | 0.797655 | 1.320043 | 0.159574 | 0.216667 | 0.37 |
| TuM3 | 10 | 0.588948 | 1.24243 | 0.146341 | 0.266667 | 0.45 |

upregulated in treated MCF7 cells

| | | | | | | |
|---|---|---|---|---|---|---|
| TuM8 | 25 | −0.51 | −1.18 | 0.429 | 0.34 | 0.38 |

siRNA-Mediated Inhibition of RLN2 and Analysis of Tamoxifen Induced Apoptosis

MCF-7 cells (ATCC) were maintained in DMEM growth media supplemented with 10% fetal bovine serum (FBS), 100 U/mL penicillin, 100 U/mL streptomycin, and 2 mM L-glutamine. MCF-7 cells were transfected with 20 nM RLN2-specific siRNA (Ambion) or control siRNA using oligofectamine transfection reagent (Invitrogen, Life Technologies). Transfected cells were maintained in DMEM with 5% DCC for 24 hrs and treated with 1 μM tamoxifen or vehicle. After 48 hrs, the treatment was terminated and the cells were maintained in DMEM with 5% DCC for 72 hrs. RLN2 silenced and control cells were treated with 1 μM Tamoxifen or equivalent quantity of vehicle for 48 hrs and subsequently, the treatment was withdrawn by changing the culture media to DMEM with 5% DCC. After 72 hrs, cells were trypsinized and stained with Annexin-V-Fluorescein and propidium iodide as recommended by the manufacturer (Roche) and the analyzed in Flow cytometer (Beckman-Coulter). The population of annexin-V positive cells was scored as the representation of the percentages of apoptotic cells.

RNA was isolated from control and RLN2 silenced MCF-7 cells at 72 hrs. Equal quantities of RNA were reverse transcribed using superscript II reverse transcriptase by oligo-T priming and RT-PCR was performed using RLN2 specific oligos to assess the efficiency of RLN2 silencing. (Oligos used for RT-PCR: RLN2-F: TGCCATCCTT CATCAACAAA, RLN2-R: CAACCAACATGGCAAC ATTT, Actin-F: CGGGAAAT CGTGCGTGACATTAAG, Actin-R: TGATCTCCTT CTGCATCCTGTCGG).

Results

Identification and Decomposition of TuMs in Breast Cancer

The inventors applied the ISA, an extension of the basic SA, to a set of ninety-six breast cancer gene expression profiles. A key parameter in the ISA is the ‘gene threshold’, a metric reflecting the stringency of co-regulation—the higher the gene threshold, the tighter the correlations between the individual genes in each TuM. When run under a series of varying gene thresholds, the ISA produces a modular decomposition of the gene expression data at different resolutions (7). FIG. 1a illustrates this concept in the form of a module tree. At low gene thresholds, a few TuMs are initially identified, where each TuM consists of a large number of loosely-correlated tumors and genes. At higher resolutions, the expression data is decomposed into a larger number of TuMs, where each TuM now contains a smaller set of tightly-correlated tumors and genes. At a gene threshold of 3.0, eight TuMs were generated; of which three were resolved from the same branch. It is worth noting that the TuMs defined by the ISA approach are distinct from the clusters defined by conventional hierarchical clustering—unlike the latter, different TuMs can share common genes and tumors (arrows in FIG. 1b)

The inventors compared the gene content of each of the eight TuMs to previous reports describing various molecular signatures in breast cancer. The first three modules (TuM1, TuM2 and TuM3) were commonly derived from a single larger module containing several genes previously reported as highly expressed in ER+ tumors, such as ESR1, GATA3, and BCL2 (1-4). Although this larger module has previously been treated as homogenous in other studies, its successful decomposition into smaller distinct units suggests that the larger module may actually comprise multiple distinct and possibly independently acting biological programs. Specifically, while both the 30-gene TuM2 and 38-gene TuM3 share substantial overlaps (˜50%) of various genes known to be regulated by ER such as BCL2 and STC2, in contrast >80% of the genes in the 34-gene TuM1 module are not found in either TuM2 or TuM3 (TuM1 is described in greater detail in the following section).

TuMs 4-8 could also be correlated to many previously defined gene expression signatures in breast cancer: TuM4 consists of a large set of genes involved in immune function, including immunoglobulin genes, T cell receptor subunits, and TNF family members (1), while TuM5, containing FBLN1, SPARC and various collagen isforms, are likely to represent contributions from the stromal cell population (1). TuM6 contained Keratin 5, Keratin 17, and SFRP1, corresponding to the expression signatures of breast cancers belonging to the Basal/ER− molecular subtype (1-4), and TuM7 contained a significant number of genes (p<10⁻⁴), belonging to the NPI-ES expression signature, previously identified as a molecular surrogate of the Nottingham Prognostic Index (8), as well as several genes involved in cell proliferation (eg MAD2L1, CDC2). TuM7 includes 85 genes and NPI-ES includes 62 genes. 16 genes were common in both gene sets. To evaluate the significance of this overlap, the inventors performed a random permutation test in the following manner: randomly select 85-gene and 62-gene sets and calculate the number of overlapping genes between the two sets; this process was then repeated 10,000 times. FIG. 5 showed that maximum overlap in the random sets is 4. Thus, the significance of overlap between TuM7 and NPI-ES can be estimated to be less than 0.0001. Finally, TuM8 contained several genes physically linked to the 17q21 locus (eg v-erb-b2, GRB7, PNMT), corresponding to a previously reported ERBB2 cluster (1-4). These results indicate that despite being an entirely unsupervised analytical approach, the ISA appears to be remarkably efficient at re-discovering many, if not all, of the major gene expression signatures previously reported for breast cancer.

TuM1 Comprises a Novel Expression Signature Associated with Apoptosis and Low Histological Grade in ER+ Tumors

Besides identifying these previously reported signatures, the ISA also discovered a novel expression signature in TuM1. TuM1 is significantly enriched in genes that have putative relationships to apoptosis (P=0.01 by the hypergeometric distribution), including programmed cell death 4, mitochondrial ribosomal protein S30 and beta-TrCP1, and also contains genes such as PCM1, recently reported to be associated with grade in breast cancer (9), cell-cell signaling genes such as GJA1 and IL6ST, and genes coding for the xenobiotic-metabolizing enzymes NAT1 and FMO5. To investigate the clinical significance of tumors exhibiting high expression of the TuM1 signature, the inventors correlated these tumors to various known clinical and histopathological parameters. To provide a basis for comparison, a similar analysis was also performed for the other TuMs as well. As can be seen in Table 1A, numerous significant associations between the TuMs and various clinical characteristics were revealed. The inventors have concentrated on the correlations exhibited by TuMs 1, 2 and 3. However, detailed discussion of the associations reported for the other TuMs is given below.

The inventors found that TuMs 1, 2 and 3 were significantly positively correlated with ER status (p<0.001; Table 1A). Consistent with this observation, tumors belonging to these three TuMs were all ER+ by standard immunohistochemistry. However, unlike TuM 2 and 3, only TuM1 exhibited a strong positive correlation with low histological grade (p<0.001, compared to p=0.024 for TuM2 and p=0.037 for TuM3), suggesting that the TuM1 expression signature might be a specific molecular feature of ER+ low-grade tumors. However, as ER+ tumors are in general associated with lower histological grade than ER− tumors (see below), the inventors also considered the possibility that the correlation between TuM1 expression and low-grade might simply be due to the predominance of ER+ tumors within these modules. To address this possibility, the inventors repeated the association studies using only ER+ tumors as the study population, in contrast to the previous analysis in Table 1A where all tumors were used. As shown in Table 1B, even after removing all non-ER+ tumors, TuM1 remained significantly correlated with low grade (p=0.001) while TuM2 and TuM3 were not (p=0.24 and p=0.21 respectively). These results indicate that the TuM1 expression signature is significantly correlated with low histological grade in a manner independent of ER status.

The TuM1 Expression Signature is Significantly Correlated with Low Histologic Grade in Two Independent Data Sets

The inventors then tested the general applicability of the TuM1 expression signature by applying it to two independent publicly available breast cancer data sets. The first data set (the “Rosetta data set”) consists of 117 breast tumors (71 ER+ tumors) profiled using oligonucleotide-based microarrays (10), while the second data set (the “Stanford data set”) consists of 122 breast tissue samples (82 ER+ tumors) profiled using cDNA microarrays (3). Of the 34 TuM1 genes identified in the present study (see Table 2), 20 and 13 genes were found on the Rosetta and Stanford microarrays respectively. Consistent with the inventor's in-house series, they found that the TuM1 signature divided the ER+ tumors in both the Rosetta and Stanford data sets into two distinct subgroups expressing high or low levels of the TuM1 expression signature, with tumors highly expressing the TuM1 signature being significantly associated with low histologic grade in both data sets (p<0.001 for both). These results indicate that the TuM1 expression signature is associated with low-histologic grade in a wide variety of patient populations, and hence it may reflect a general molecular feature of breast cancer. Interestingly, in both data sets, most, but not all, of the previously-defined ‘Luminal A’ subtype tumors (3) expressed high levels of the TuM1 signature, even though only one of the 34 genes in TuM1 (NAT1) has previously been reported to be expressed in this tumor subtype.

As clinical follow-up data was also available for the Rosetta and Stanford patient cohorts, the inventors tested the ability of the TuM1 signature to predict clinical outcome in these two patient populations. They found that in the Stanford series, patients with TuM1-expressing ER+ tumors exhibited better survival outcomes compared to patients with ER+ tumors where TuM1 was not expressed (p=0.0001 for overall survival; p=0.0036 for relapse-free survival, FIG. 2a). In contrast, in the Rosetta series, patients with TuM1-expressing ER+ tumors did not exhibit an improved clinical outcome compared to patients with ER+ tumors where TuM1 was not expressed (p=0.34). A possible reason explaining this difference between these two populations may lie in the distinct clinical characteristics of the two cohorts: While the Rosetta series comprises early stage (Stage I) patients that in general did not receive any systemic adjuvant therapy, the Stanford series consists primarily of later stage patients with locally advanced disease who received adjuvant endocrine treatment after surgery (if their tumors were ER+). It is thus possible that the presence of the TuM1 signature may reflect a tumor's sensitivity to adjuvant treatment rather than a tumor's intrinsic tendency to metastasize (see Discussion).

Correlation Analysis Between Different TuMs Reveals an Unexpected Relationship Between ERBB2+ Tumors and the Immune System

A major strength of SA is the ability to reveal higher-order correlations between the different modules (5). In the context of tumor biology, this can be highly useful in identifying relationships between the various TuMs, and to determine if the expression of the different molecular signatures within a particular tumor are occurring in an independent or non-independent fashion. The inventors calculated correlation values between the different TuMs (see below), and depicted the results as a heat-map illustrating the relationships of the different tumors across the TuMs (FIG. 3). For example, TuMs 1, 2 and 3 display a highly overlapping (but not identical) ‘tumor signature’ (FIG. 3A), indicating that tumors expressing the TuM1 signature are likely to express the TuM2 and TuM3 signatures as well. Similarly, TuM7, the NPI-ES/cellular proliferation module, was positively correlated with TuM6 (the ‘basal’ module) but negatively correlated with TuM1 (FIG. 3B). These findings are consistent with previously known traits of breast cancers—for example, it is known that tumors of the ER− or ‘basal’ subtype typically have a high histologic grade and increased expression of proliferation markers such as Ki67 (1-4). However, in addition to these expected findings, the inter-TuMs correlation analysis revealed an unexpected finding—specifically, the TuM4 ‘immune’ module was found to be correlated with the ERBB2+ module TuM8 (FIG. 3C), at a correlation strength comparable to the other relationships highlighted in FIG. 3. This correlation suggests the presence of substantial cross-talk between immune cells and tumor cells of the ERBB2+ molecular subtype, and is further addressed in the discussion. Notably, tumors exhibiting a common TuM4 (+) TuM8 (+) ‘tumor signature’ were weakly but significantly correlated with increased lymphovascular invasion (LVI) (p=0.03; Chi-square analysis), unlike tumors that were either TuM4 (+) or TuM8 (+) alone. This result suggests that tumors expressing both the TuM4 and TuM8 signatures may be associated with clinical characteristics distinct from tumors that express either signature in isolation.

Association Between TuMs and Clinical Parameters

A tumor module is associated with a set of tumors. The significance of each tumor is characterized by a score. A positive or negative score indicates that in this tumor the genes are upregulated or downregulated. Here the inventors only study tumors with positive score because tumors with negative score are insufficient (only three modules had tumors with negative scores; see FIG. 6). They found that certain tumors with low tumor score are clearly apart from others (those in the rectangle). These tumors were treated as “low confidence” samples and removed them from subsequent correlation analysis (Table 1A).

Statistical approaches were then used to discover the clinical significance of these transcriptional modules. The results revealed a number of significant associations between modules and clinical characteristics. Overall, the inventor's results (especially under stringent significance thresholds: p<0.01, Table 1A) suggest that only ER/PR status and tumor grade are likely to be associated with gene expression data, which was also observed by ref. 4. TuM4, the immune cluster, was negatively correlated with ER and marginally positively correlated with high grade (p=0.02). This result is consistent with the report that Immunoglobin genes comprised the majority of ‘ER−’ genes (Iwko et al., 2002). TuM5, the predominantly stromal cell cluster, was not associated with any clinical parameters. As expected, TuM6 and TuM8, representing ER−/Basal and ERBB2+ respectively, were significantly negatively correlated with ER (p<0.001). For the TuM8 (ERBB2+), 14 tumors for which ERBB2 IHC had been performed were all ERBB2+ by IHC as well. TuM7, the cell proliferation cluster, is significantly correlated with high histological grade but not correlated with ER status.

Correlations Between TuMs

Followed by the instructions given by Bergmann et al., 2004, the inventors calculated the correlation values between TuMs, corresponding to FIG. 3.

TuM1 Expression is Associated with Low Histologic Grade

Using multivariate analysis, we tested if the correlation between TuM1 expression and low tumor grade was simply a consequence of their association with ER status, or if the association between TuM1 expression and low tumor grade was independent of ER. In this analysis, TuM1 expression was correlated with grade independently of ER (p<0.001), but the association of TuM2, another tumor module, with low grade was not (p=0.9) (Table 5)

The TuM1 Module is Downregulated by Tamoxifen Treatment in vitro

The observation that TuM1 is expressed in a subset of ER+ tumors raises the possibility that expression of this module may depend, at least in some part, on ER activity and signaling. To investigate the relationship between TuM1 expression and ER signaling, we tested the responsiveness of TuM1 to ER activity using an in vitro system. First, by profiling a set of breast and gastric cancer cell lines, we found that the TuM1 module was overexpressed in the ER+ breast cancer cell line MCF7 (FIG. 11). Second, we treated MCF7 cells with tamoxifen, an inhibitor of ER, and using gene set enrichment analysis (GSEA, 16) further discovered that TuM1 was significantly downregulated in tam-treated MCF7 cell lines compared to controls (FDR=0.05). As a control, none of the other TuMs were affected by tamoxifen treatment with the exception of TuM2, which was marginally correlated with tamoxifen treatment (FDR=0.19). The details of this analysis are given in the Materials and Methods. This result suggests that at least in vitro, TuM1 expression may be dependent on active ER signaling, and may thus represent a ‘molecular signature’ of ER activity.

A Possible Association Between TuM1 Expression and Clinical Outcome

Our finding that expression of the TuM1 module is dependent on active ER signaling made us investigate if the presence of this module in primary tumors might function as a molecular biomarker for active ER activity, and identify tumors that are likely to respond to tamoxifen or other anti-hormonal treatments. We tested the prognostic ability of TuM1 in three data sets. In the first data set from Stanford University, in a multivariate analysis of TuM1, grade, age, lymph node and tumor size, TuM1 behaved as an independent predictor of survival outcome, while grade did not, demonstrating that TuM1 is more directly prognostic of patient survival than grade status alone (Table 6). Second, we tested the Ma data set, which comprises a set of pre-selected tamoxifen responsive and resistant ER+ tumors (28). Once again, TuM1-overxpressing patients exhibited significantly better outcome than low TuM1 patients (p=0.048, FIG. 12b). By multivariate Cox regression analysis, TuM1 was the sole independent prognosis factor (p=0.03; Table 6); as grade, tumor size, node and age are controlled in the Ma patient cohort (28). This observation was also tested using Gene Set Enrichment Analysis (GSEA) which confirmed that TuM1 expression was significantly associated with tamoxifen response (p=0.024;). Third, the prognostic ability of TuM1 was tested on the Uppsala set, an independent patient cohort of sixty-seven ER+ patients who received tamoxifen as monotherapy (29). Once again, patients with TuM1 expressing tumors experienced significantly improved overall survival outcomes compared to low TuM1-expressing patients (p=0.025, FIG. 12c). By multivariate Cox regression analysis, TuM1 remained significantly associated with survival (p=0.024); while grade, tumor size, and lymph node status did not (Table 6).

Knockdown of Relaxin 2, a TuM1 Module Gene, Decreases MCF7 Response to Tamoxifen

To functionally investigate the association of the TuM1 signature with a tumor's response to anti-hormonal treatment, the role of a representative TuM1 gene, Relaxin2 (RLN2) was assessed in a ER+ breast cancer cellular model. RLN2 gene was silenced in MCF-7 cell line by siRNA mediated knockdown. The RLN2 silenced and control cells were treated with 1 μm tamoxifen for 48 hrs and the percentage of apoptotic cells were analyzed after 72 hrs. The flow cytometric analysis revealed that about 73% of the cells in the tamoxifen treated control MCF-7 cells were annexin-V-staining positive whereas, in the RLN2 silenced MCF-7 population, about 23% of the cells were apoptotic. It shows that high level expression of RLN2 somehow confers tamoxifen responsiveness in the breast cancer cell line model as evidenced by the reduced Tamoxifen sensitivity of RLN2 silenced cell lines. The unknown molecular mechanisms by which TuM1 genes confer responsiveness to anti-hormonal treatment merit a detailed study.

Discussion

The inventors employed a recently described analytical methodology, Signature Analysis, to characterize an in-house data set of breast tumor expression profiles. In addition to rediscovering many previously described gene expression signatures in breast cancer, the SA identified a novel gene expression signature (TuM1) that was significantly enriched in genes related to apoptosis and correlated with low histologic grade in three independent data sets. It is worth noting that the association of the TuM1 signature with low histologic grade was demonstrated to be independent of ER status. The TuM1 signature is thus distinct from previously reported expression signatures for low histological grade, which have tended to comprise genes related to ER status such as GATA3 (4), which may reflect the well-known observation that ER negative tumors tend to be high-grade.

Many of co-regulated genes identified in TuM1 have been linked to apoptosis. Among them, programmed cell death 4 (PDCD4) has been shown to inhibit the growth of tumor cells (11), beta-TrCP1 (BTRC; also known as Fbwla or FWD1), a component of the SCF (SKP1-cullin-F-box) ubiquitin protein ligase complex, functions in multiple transcriptional programs by activating the NF-kappaB (NFκB) pathway, which in turn represses cell proliferation (12), and heat shock 70 kDa protein 2 (HSPA2) may provide cellular protection from apoptosis (13). Intriguingly, inactivation of PDCD4 in human cancers has also been reported to cause decreased sensitivity to geldanamycin cytotoxicity, as well as to tamoxifen in breast cancer in vitro (14), while NAT1, another TuM1 gene, has been reported as an independent prognostic factor of breast cancer relapse and a potential predictor of tamoxifen response (15). These latter observations suggest that the TuM1 signature will function as a predictive signature for response to hormonal therapies in breast cancer. Consistent with this possibility, the TuM1 signature was strongly associated with clinical outcome in patient populations receiving adjuvant hormonal treatment (the Stanford cohort), but was not associated with clinical outcome in patient populations that did not receive such treatment (the Rosetta cohort). Notably, it has also been recently reported that breast tumors with overexpression of apoptosis-related genes can display enhanced sensitivity to chemotherapy (16).

In addition to identifying TuM1, the SA also allowed the inventors to define correlations between the various TuMs to explore the higher-order regulatory relationships between these co-regulated gene groups. They discovered a striking positive correlation between TuM4, containing immune-related genes, and TuM8, containing ERBB2 related genes and hence representative of the ERBB2+ tumor subtype. This result raises the possibility that substantial cross-talk may occur between ERBB2+ tumor cells and cells of the immune system. At the present moment, the inventors can only speculate on the possible molecular mechanisms underlying this process. A potential clue, however, can potentially be found by examining the gene expression data. Among the TuM4 genes, GBP1 and ISG20 have been previously reported as target genes of NF-kappaB (17, 18), a key component of the immune response pathway (19) that regulates the expression of inflammatory cytokines, chemokines, immunoreceptors, and cell adhesion molecules. Moreover, Biswas et al has recently reported that activated NFκB can be found predominantly in the ER-neg/ERBB2-positive subgroup of breast tumors (20). Thus, the inventors believe that the positive relationship between TuM4 (immune response) and TuM8 (ERBB2) may be due at least in part to the activation of NFκB specifically in ERBB2+ tumor cells, which then mediates the activation of the immune response. Intriguingly, the inventors found that tumors expressing both the TuM4 and TuM8 signatures were significantly correlated with LVI. As such, cross-talk between tumor cells and the immune system may contribute to the ability of these tumors to exhibit enhanced angiogensis and tendency for metastasis, both of which have been related to NFκB activity (21).

In conclusion, the inventors have demonstrated the feasibility of performing SA on cancer expression data, and shown that the SA analysis can yield novel biological findings, even for data sets that have received substantial prior analysis. SA thus provides a powerful alternative method to cluster genes and to integrate external clinical information with gene expression data. Furthermore, the TuMs defined by SA further our understanding of the higher-level molecular relationships occurring in breast cancer and enable important diagnosis, prognosis and treatment regimen decisions to be made.

Table 1A. Association between tumor modules and clinical parameters using both Chi-square analysis and Hypergeometric probability density function analysis. Only the significant p-values (<0.05) confirmed by both analyses were highlighted. The value in bold indicate the most significant correlations (<0.001). LN: lymph node, ER: estrogen receptor status; PR: progesterone receptor; and LVI: lymphovascular invasion.

TABLE 1A
Correlations between Modules and Clinical Characteristics.

| | Age | Size | Grade | | | | |
|---|---|---|---|---|---|---|---|
| | (≦/>55) | (≦/>3 cm) | (1, 2) vs. 3 | LN | ER | PR | LVI |
| TuM1 (Low | | 0.0152 (≦3*) | <0.001 (1, 2) | | <0.001 | 0.0107 | 0.0152 |
| Grade) | | | | | (+) | (+) | (−) |
| TuM2 | | | 0.0242 (1, 2) | | <0.001 | 0.0021 | |
| (ER+/Luminal) | | | | | (+) | (+) | |
| TuM3 (ER+ II) | | | 0.0371 (1, 2) | | <0.001 | 0.0015 | |
| | | | | | (+) | (+) | |
| TuM4 (Immune) | | | 0.0212 (3) | | 0.0044 | | |
| | | | | | (−) | | |
| TuM5 (Stroma) | | | | | | | |
| TuM6 (ER−/ | | | | 0.0236 | <0.001 | 0.0098 | |
| Basal) | | | | (+) | (−) | (−) | |
| TuM7 (Cell | | | <0.001 (3) | | | | |
| Proliferation) | | | | | | | |
| TuM8 (ERBB2+) | | | | | <0.001 | <0.001 | |
| | | | | | (−) | (−) | |

*The parameter in the ( ) indicates the direction of correlation with the TuM. For example, TuM2 is positively correlated with high grade (3) and ER-neg (−); while TuM3 is positively correlated with smaller tumor size (≦3), low grade (1, 2), ER-pos (+), PR-pos (+) and LVI-neg (−).

Table 1B. Associations between TuM1, 2, 3 and histological grade in ER+ tumors only. There are two columns for each module: the 1^{st} column is the tumor belonging to the tumor module; and the 2^{nd} column represents all remaining ER+ tumors.

TABLE 1B
Correlation between TuMs 1, 2, 3 and tumor grade within ER+
tumors.

| | | | |
|---|---|---|---|
| | TuM1 (Low | TuM2 (ER+/ | TuM3 |
| | Grade) | Luminal) | (ER+ II) |
| Grade | P = 0.0001 | P = 0.2395 | P = 0.2123 |

| | | | | | | |
|---|---|---|---|---|---|---|
| 1 & 2 | 12 | 12 | 12 | 12 | 8 | 16 |
| 3 | 2 | 30 | 11 | 21 | 6 | 26 |

TABLE 2
Co-regulated genes in TuM1

| Probe | Gene Name | Unigene |
|---|---|---|
| 218613_at | hypothetical protein DKFZp761K1423 | Hs.236438 |
| 203355_s_at | ADP-ribosylation factor guanine nucleotide factor 6 | Hs.408177 |
| 202731_at | programmed cell death 4 (neoplastic transformation inhibitor) | Hs.257697 |
| 214440_at | N-acetyltransferase 1 (arylamine N-acetyltransferase) | Hs.458430 |
| 203404_at | armadillo repeat protein ALEX2 | Hs.48924 |
| 202174_s_at | pericentriolar material 1 | Hs.348501 |
| 217838_s_at | Enah/Vasp-like | Hs.241471 |
| 219455_at | hypothetical protein FLJ21062 | Hs.276466 |
| 221946_at | hypothetical protein MGC29761 | Hs.414028 |
| 222314_x_at | Homo sapiens, clone IMAGE: 5759947, mRNA | Hs.437867 |
| 211596_s_at | leucine-rich repeats and immunoglobulin-like domains 1 | Hs.166697 |
| 211538_s_at | heat shock 70 kDa protein 2 | Hs.432648 |
| 214705_at | InaD-like protein | Hs.436450 |
| 218398_at | mitochondrial ribosomal protein S30 | Hs.124165 |
| 201667_at | gap junction protein, alpha 1, 43 kDa (connexin 43) | Hs.74471 |
| 215300_s_at | flavin containing monooxygenase 5 | Hs.396595 |
| 209884_s_at | solute carrier family 4, sodium bicarbonate cotransporter, member 7 | Hs.250072 |
| 212196_at | interleukin 6 signal transducer (gp130, oncostatin M receptor) | Hs.71968 |
| 200648_s_at | glutamate-ammonia ligase (glutamine synthase) | Hs.442669 |
| 214519_s_at | relaxin 2 (H2) | Hs.127032 |
| 219114_at | g20 protein | Hs.21050 |
| 206081_at | solute carrier family 24 (sodium/potassium/calcium exchanger), member 1 | Hs.173092 |
| 214430_at | galactosidase, alpha | Hs.69089 |
| 221562_s_at | sirtuin (silent mating type information regulation 2 homolog) 3 (S. cerevisiae) | Hs.511950 |
| 218149_s_at | hypothetical protein DKFZp434K1210 | Hs.32352 |
| 214087_s_at | myosin binding protein C, slow type | Hs.169849 |
| 213933_at | prostaglandin E receptor 3 (subtype EP3) | Hs.27860 |
| 215014_at | Homo sapiens mRNA; cDNA DKFZp547P042 (from clone DKFZp547P042) | Hs.232127 |
| 203143_s_at | KIA0040 gene product | Hs.368916 |
| 204901_at | beta-transducin repeat containing | Hs.226434 |
| 209123_at | quinoid dihydropteridine reductase | Hs.75438 |
| 213832_at | Homo sapiens clone 24405 mRNA sequence | Hs.23729 |
| 207519_at | solute carrier family 6 (neurotransmitter transporter, serotonin), member 4 | Hs.448453 |

TABLE 2a

| Rank | Probe Set I | Gene Title | Gene Symbol | UniGene ID | LocusLink |
|---|---|---|---|---|---|
| 1 | 219455_at | hypothetical protein FLJ21062 | FLJ21062 | Hs.521012 | 79846 |
| 2 | 214519_s_{—} | relaxin 2 | RLN2 | Hs.127032 | 6019 |
| 3 | 212196_at | Interleukin 6 signal transducer (gp130, oncostatin M receptor) | IL6ST | Hs.532082 | 3572 |
| 4 | 213933_at | Prostaglandin E receptor 3 (subtype EP3) | PTGER3 | Hs.445000 | 5733 |
| 5 | 201667_at | gap junction protein, alpha 1, 43 kDa (connexin 43) | GJA1 | Hs.74471 | 2697 |
| 6 | 215300_s_{—} | flavin containing monooxygenase 5 | FMO5 | Hs.303476 | 2330; 10694 |
| 7 | 213832_at | Clone 24405 mRNA sequence | — | Hs.23729 | |
| 8 | 207519_at | solute carrier family 6 (neurotransmitter transporter, serotonin), member 4 | SLC6A4 | Hs.448453 | 6532 |
| 9 | 209123_at | quinoid dihydropteridine reductase | QDPR | Hs.75438 | 5860 |
| 10 | 202731_at | programmed cell death 4 (neoplastic transformation inhibitor) | PDCD4 | Hs.232543 | 27250; 282997 |
| 11 | 200648_s_{—} | glutamate-ammonia ligase (glutamine synthase) | GLUL | Hs.518525 | 2752 |
| 12 | 214087_s_{—} | myosin binding protein C, slow type | MYBPC1 | Hs.506502 | 4604 |
| 13 | 202174_s_{—} | pericentriolar material 1 | PCM1 | Hs.491148 | 5108 |
| 14 | 211596_s_{—} | leucine-rich repeats and immunoglobulin-like domains 1 /// leucine-rich | LRIG1 | Hs.518055 | 26018 |
| | | repeats and immunoglobulin-like domains 1 | | | |
| 15 | 211538_s_{—} | heat shock 70 kDa protein 2 | HSPA2 | Hs.432648 | 3306 |
| 16 | 203143_s_{—} | KIAA0040 | KIAA0040 | Hs.518138 | 9674 |
| 17 | 214430_at | galactosidase, alpha | GLA | Hs.69089 | 2717 |
| 18 | 203404_at | armadillo repeat containing, X-linked 2 | ARMCX2 | Hs.48924 | 9823 |
| 19 | 214440_at | N-acetyltransferase 1 (arylamine N-acetyltransferase) | NAT1 | Hs.155956 | 9 |
| 20 | 204901_at | beta-transducin repeat containing | BTRC | Hs.500812 | 8945 |
| 21 | 209884_s_{—} | solute carrier family 4, sodium bicarbonate cotransporter, member 7 | SLC4A7 | Hs.250072 | 9497 |
| 22 | 206081_at | solute carrier family 24 (sodium/potassium/calcium exchanger), member 1 | SLC24A1 | Hs.173092 | 9187 |
| N/A | 219114_at | chromosome 3 open reading frame 18 | C3orf18 | Hs.517860 | 51161 |
| N/A | 221946_at | chromosome 9 open reading frame 116 | C9orf116 | Hs.414028 | 138162 |
| N/A | 217838_s_{—} | Enah/Vasp-like | EVL | Hs.125867 | 51466 |
| N/A | 222314_x_{—} | Homo sapiens, clone IMAGE: 5759947, mRNA | — | Hs.437867 | |
| N/A | 214705_at | InaD-like (Drosophila) | INADL | Hs.478125 | 10207 |
| N/A | 218398_at | mitochondrial ribosomal protein S30 | MRPS30 | Hs.124165 | 10884 |
| N/A | 215014_at | MRNA; cDNA DKFZp547P042 (from clone DKFZp547P042) | — | Hs.485819 | |
| N/A | 203355_s_{—} | pleckstrin and Sec7 domain containing 3 | PSD3 | Hs.434255 | 23362 |
| N/A | 221562_s_{—} | sirtuin (silent mating type information regulation 2 homolog) 3 (S. cerevisiae) | SIRT3 | Hs.549124 | 23410 |
| N/A | 218149_s_{—} | zinc finger protein 395 | ZNF395 | Hs.435535 | 55893 |

TABLE 3
Patient and Sample Information
Clinical information for Breast Tumors

| Sample ID | Age | Size (mm) | Grade | LN | ER | PR | LVI | cerbB2 |
|---|---|---|---|---|---|---|---|---|
| 980058 | 72 | 45 | 3 | 0 of 12 | pos | pos | No | |
| 980177 | 75 | 26 | 2 | 6 of 13 | pos | pos | yes | neg |
| 980178 | 69 | 32 | 3 | 2 of 15 | pos | neg | No | neg |
| 980193 | 49 | 25 | 3 | 3 of 23 | neg | neg | No | |
| 980194 | 58 | 50 | 3 | 25 of 32 | neg | neg | yes | |
| 980197 | 55 | 30 | 3 | 2 of 4 | pos | pos | yes | |
| 980203 | 44 | 15 | 1 | 0 of 11 | pos | pos | No | |
| 980208 | 42 | 25 | 3 | 5 of 20 | pos | pos | No | |
| 980214 | 49 | 60 | 2 | 5 of 13 | pos | neg | No | pos 2+ |
| 980215 | 50 | 30 | 2 | 8 of 23 | pos | neg | No | |
| 980216 | 65 | 45 | 2 | 5 of 20 | neg | neg | No | |
| 980217 | 50 | 30 | 2 | 7 of 12 | pos | neg | yes | |
| 980220 | 40 | 37 | 2 | 0 of 5 | pos | pos | yes | |
| 980221 | 33 | 65 | 3 | 1 of 13 | pos | pos | No | neg |
| 980238 | 62 | 20 | 3 | 7 of 21 | neg | neg | No | |
| 980247 | 35 | 45 | 3 | 1 of 19 | neg | neg | yes | pos |
| 980256 | 46 | 36 | 3 | 1 of 12 | neg | neg | No | pos |
| 980261 | 60 | 15 | 2 | 0 of 9 | pos | neg | No | |
| 980278 | 64 | 40 | 3 | 14 of 20 | pos | neg | yes | pos 2+ |
| 980285 | 49 | 40 | 3 | 1 of 7 | neg | neg | yes | pos |
| 980288 | 45 | 60 | 3 | 13 of 15 | pos | neg | yes | pos |
| 980315 | 59 | 45 | 3 | 0 of 19 | neg | neg | yes | |
| 980333 | 51 | 40 | 3 | 2 of 7 | pos | pos | No | |
| 980335 | 33 | 3 | 3 | 3 of 7 | neg | neg | yes | pos |
| 980338 | 55 | 30 | 3 | 0 of 7 | neg | neg | No | |
| 980346 | 52 | 20 | 3 | 0 of 4 | pos | pos | possible | 3+ |
| 980353 | 58 | 45 | 3 | 0 of 25 | neg | neg | No | |
| 980373 | 77 | 30 | 3 | 0 of 14 | neg | neg | No | |
| 980380 | 56 | | | 0 of 6 | neg | neg | | |
| 980383 | 64 | 30 | 2 | 0 of 16 | pos | pos | No | |
| 980391 | 56 | 20 | 2 | 0 of 7 | pos | pos | No | |
| 980395 | 68 | 30 | 3 | 1 of 10 | neg | neg | yes | |
| 980396 | 66 | 35 | 3 | 10 of 12 | neg | neg | yes | |
| 980403 | 73 | 30 | 3 | 0 of 9 | pos | pos | possible | |
| 980404 | 46 | 30 | 2 | 1 of 5 | pos | pos | yes | |
| 980409 | 48 | 15 | 2 | 0 of 19 | pos | neg | No | |
| 980411 | 69 | 30 | 2 | 0 of 9 | neg | neg | No | |
| 980434 | 73 | 30 | 3 | 0 of 16 | pos | pos | No | |
| 980441 | 66 | 30 | 3 | 4 of 14 | neg | neg | yes | |
| 990075 | 66 | 25 | 3 | 5 of 21 | pos | pos | yes | |
| 990082 | 49 | 34 | 2 | 3 of 16 | pos | pos | No | |
| 990107 | 50 | 40 | 1 | 1 of 18 | pos | neg | yes | |
| 990113 | 70 | 90 | 3 | 11 of 15 | pos | pos | No | |
| 990115 | 38 | 28 | 3 | 9 of 10 | pos | pos | yes | |
| 990123 | 54 | 55 | 3 | 7 of 11 | pos | pos | No | |
| 990134 | 43 | 40 | 3 | 0 of 19 | neg | neg | No | |
| 990148 | 60 | 40 | 2 | 6 of 19 | pos | neg | yes | |
| 990174 | 55 | 45 | 2 | 3 of 24 | neg | neg | yes | |
| 990223 | 52 | 5 | 3 | 1 of 21 | pos | neg | No | |
| 990262 | 68 | 40 | 3 | 4 of 14 | neg | neg | No | |
| 990299 | 58 | 55 | 3 | 7 of 17 | neg | neg | possible | |
| 990375 | 38 | 15 | 1 | 0 of 10 | pos | neg | No | |
| 2000104 | 59 | | | | pos | neg | | pos |
| 2000171 | 50 | 25 | 2 | 0 of 9 | neg | neg | No | pos |
| 2000209 | 58 | 50 | 3 | 0 of 7 | pos | neg | No | pos |
| 2000210 | 50 | 40 | 3 | 3 of 6 | neg | neg | yes | pos |
| 2000215 | 50 | 15 | 2 | 1 of 21 | pos | pos | No | |
| 2000220 | 52 | 60 | 3 | 30 of 34 | pos | neg | yes | pos |
| 2000237 | 43 | 47 | 3 | 23 of 40 | pos | pos | yes | pos |
| 2000272 | 49 | 30 | 3 | 1 of 16 | pos | neg | yes | |
| 2000274 | 40 | 35 | 3 | 10 of 23 | pos | pos | yes | |
| 2000287 | 53 | 40 | 3 | 0 of 8 | neg | neg | possible | pos |
| 2000320 | 67 | 20 | 3 | 20 of 21 | neg | neg | yes | pos |
| 2000376 | 65 | | 3 | 8 of 23 | neg | neg | yes | |
| 2000399 | 44 | 40 | 2 | 0 of 8 | neg | neg | No | pos |
| 2000401 | 51 | 50 | 3 | 2 of 6 | neg | pos | No | |
| 2000422 | 51 | 63 | 3 | 3 of 7 | pos | pos | No | neg |
| 2000500 | 44 | 75 | 3 | 6 of 6 | neg | neg | yes | |
| 2000593 | 60 | 41 | 3 | 0 of 15 | neg | neg | No | pos |
| 2000597 | 57 | 40 | 2 | 0 of 12 | pos | neg | possible | pos 3+ |
| 2000609 | 62 | 70 | 2 | 17 of 17 | pos | pos | yes | pos |
| 2000638 | 60 | 40 | 1 | 0 of 15 | pos | neg | No | Intermediate |
| 2000641 | 47 | 60 | 3 | 16 of 24 | neg | neg | yes | pos |
| 2000651 | 45 | 41 | 2 | 3 of 5 | pos | pos | yes | |
| 2000652 | 56 | 25 | 3 | 6 of 21 | neg | neg | No | pos |
| 2000675 | 78 | 55 | 3 | 16 of 16 | neg | neg | yes | pos |
| 2000683 | 72 | 35 | 2 | 0 of 17 | pos | pos | No | neg |
| 2000709 | 45 | 30 | 3 | 0 of 16 | neg | neg | No | pos |
| 2000731 | 68 | 51 | 3 | 1 of 29 | pos | neg | No | pos |
| 2000759 | 57 | 7 | 3 | 0 of 12 | neg | neg | No | pos |
| 2000768 | 39 | 40 | 3 | 0 of 17 | pos | pos | No | pos 2+ |
| 2000775 | 51 | 25 | 2 | 0 of 12 | pos | neg | No | neg |
| 2000779 | 48 | 55 | 3 | 0 of 14 | pos | neg | No | neg |
| 2000787 | 57 | 60 | 3 | 0 of 9 | pos | pos | yes | pos 3+ |
| 2000804 | 39 | 40 | 3 | 5 of 21 | pos | pos | yes | neg |
| 2000813 | 60 | 23 | 3 | 16 of 17 | neg | neg | yes | pos |
| 2000818 | 52 | 10 | 2 | 0 of 11 | pos | neg | No | pos 2+ |
| 2000829 | 51 | 45 | 2 | 10 of 10 | neg | neg | yes | pos |
| 2000880 | 55 | 15 | 2 | 0 of 26 | neg | neg | No | |
| 2000948 | 56 | 35 | 3 | 4 of 22 | pos | neg | yes | |
| 20020021 | 64 | 38 | 3 | 0 of 13 | pos | neg | yes | |
| 20020051 | 38 | 50 | 3 | 1 of 25 | pos | pos | No | pos 2+ |
| 20020056 | 71 | 20 | 1 | 2 of 17 | pos | neg | No | pos 2+ |
| 20020071 | 58 | 28 | 3 | 0 of 16 | pos | pos | No | pos 2+ |
| 20020090 | 60 | 45 | 3 | 19 of 27 | neg | neg | yes | pos 3+ |
| 20020160 | 86 | 120 | 3 | 0 of 10 | pos | pos | No | neg |

LN: lymph node;
ER: estrogen receptor;
PR: progesterone receptor;
LVI: lymphovascular invasion

TABLE 4
Correlation values of FIG. 3. The top
correlations values are highlighted in bold.

| | 1 | 2 | 3 | 4 | 5 | 6 | 7 | 8 |
|---|---|---|---|---|---|---|---|---|
| TuM1 | X | | | | | | | |
| (Low Grade) | | | | | | | | |
| TuM2 | 0.65 | X | | | | | | |
| (ER+/Luminal) | | | | | | | | |
| TuM3 (ER+ II) | 0.42 | 0.76 | X | | | | | |
| TuM4 | −0.01 | 0.07 | 0.11 | X | | | | |
| (Immune) | | | | | | | | |
| TuM5 (Stroma) | 0.26 | 0.24 | 0.13 | 0.29 | X | | | |
| TuM6 | −0.01 | −0.02 | 0.06 | 0.12 | 0.08 | X | | |
| (ER−/Basal) | | | | | | | | |
| TuM7 (Cell | −0.16 | 0.06 | 0.01 | 0.17 | −0.02 | 0.39 | X | |
| Proliferation) | | | | | | | | |
| TuM8 | 0.04 | 0.1 | 0.07 | 0.31 | 0.29 | 0.14 | 0.18 | X |
| (ERBB2+) | | | | | | | | |

S, Ihmels J, Barkai N. Similarities and differences in genome-wide expression data of six organisms. PLoS Biol. 2(1): E9, 2004.

Table 5. Correlation between grade and TuMs and other clinical parameters in breast cancer by using linear regression multivariate analysis (SPSS). Besides TuM1, only PR is marginally correlated with grade. The positive regression coefficient means the variable is associated with low grade.

TABLE 5

| | | | 95% Confidence | |
|---|---|---|---|---|
| | | | Interval | |
| | | | for Regression | |
| | P- | Regression | Coefficient | |
| Variable | Value | Coefficient | Lower Bound | Upper Bound |
| TUM1 | <0.001 | 0.783 | 0.404 | 1.162 |
| TUM2 | 0.898 | −0.025 | −0.418 | 0.367 |
| TUM3 | 0.586 | −0.111 | −0.516 | 0.294 |
| TuM4 | 0.353 | −0.125 | −0.391 | 0.141 |
| TuM5 | 0.426 | 0.120 | −0.179 | 0.420 |
| TuM6 | 0.405 | 0.127 | −0.174 | 0.427 |
| TuM7 | 0.192 | −0.184 | −0.462 | 0.094 |
| TuM8 | 0.337 | −0.137 | −0.420 | 0.146 |
| AGE | 0.197 | 0.006 | −0.003 | 0.016 |
| SIZE | 0.317 | 0.003 | −0.003 | 0.009 |
| NODE | 0.106 | 0.183 | −0.040 | 0.406 |
| ER | 0.091 | −0.255 | −0.551 | 0.041 |
| PR | 0.020 | 0.315 | 0.052 | 0.579 |

Table 6: Multivariate analysis of risk factors for death (Uppsala and Stanford) or metastasis (Ma) as the first event. Parameters found to be significant (P<0.05) in the COX proportional hazard model are shown in bold.

TABLE 6

| Ma | p-value | Hazard ratio (95% CI) | Uppsala | p-value | Hazard ratio (95% CI) |
|---|---|---|---|---|---|
| TUM1 | 0.030 | 0.4 (0.175-0.913) | TUM1 | 0.024 | 0.27 (0.087-0.838) |
| SIZE | 0.150 | 1.307 (0.908-1.883) | SIZE | 0.534 | 1.016 (0.967-1.067) |
| NODE(2) | 0.532 | 1.308 (0.564-3.037) | P53 | 0.998 | 0.999 (0.307-3.243) |
| NODE(1) | 0.709 | 1.321 (0.306-5.704) | NODE(2) | 0.065 | 0.307 (0.088-1.075) |
| NODE | 0.809 | | NODE(1) | 0.983 | |
| GRADE | 0.568 | 1.274 (0.555-2.923) | NODE | 0.181 | |
| AGE | 0.309 | 0.977 (0.935-1.021) | GRADE | 0.853 | 0.92 (0.38-2.226) |
| | | | AGE | 0.016 | 1.058 (1.01-1.108) |

| Stanford | p-value | Hazard ratio (95% CI) |
|---|---|---|
| TUM1 | 0.003 | 0.067 (0.012-0.388) |
| SIZE | 0.113 | 2.206 (0.83-5.868) |
| NODE | 0.439 | 0.801 (0.456-1.406) |
| METATASIS | 0.007 | 5.822 (1.633-20.75) |
| GRADE | 0.090 | 2.094 (0.892-4.917) |
| AGE | 0.577 | 0.989 (0.951-1.028) |

### REFERENCE

1. Perou, C. M., T. Sorlie, M. B. Eisen, v. d. R. M., S. S. Jeffrey, C. A. Rees, J. R. Pollack, D. T. Ross, H. Johnsen, L. A. Akslen, O. Fluge, A. Pergamenschikov, C. Williams, S. X. Zhu, P. E. Lonning, A. L. Borresen-Dale, P. O. Brown, and D. Botstein. Molecular Portraits of Human Breast Tumors. Nature, 406: 747-752, 2000.

2. Sorlie, T., C. M. Perou, R. Tibshirani, T. Aas, S. Geisler, H. Johnsen, T. Hastie, M. B. Eisen, M. van de Rijn, S. S. Jeffrey, T. Thorsen, H. Quist, J. C. Matese, P. O. Brown, D. Botstein, P. E. Lonning, and A. L. Borresen-Dale. Gene Expression Patterns of Breast Carcinomas Distinguish Tumor Subclasses with Clinical Implications. Proc Natl Acad Sci U S A., 98: 10879-10874, 2001.

3. Sorlie T, Tibshirani R, Parker J, Hastie T, Marron J S, Nobel A, Deng S, Johnsen H, Pesich R, Geisler S, Demeter J, Perou C M, Lonning P E, Brown P O, Borresen-Dale A L, Botstein D. Repeated observation of breast tumor subtypes in independent gene expression data sets. Proc Natl Acad Sci U S A. 100(14): 8418-23, 2003.

4. Sotiriou C, Neo S Y, McShane L M, Korn E L, Long P M, Jazaeri A, Martiat P, Fox S B, Harris A L, Liu E T. Breast cancer classification and prognosis based on gene expression profiles from a population-based study. Proc Natl Acad Sci U S A., 100(18): 10393-8, 2003.

5. Yu K, Lee C H, Tan P H, Tan P. Conservation of Breast Cancer Molecular Subtypes and Transcriptional Patterns of Tumor Progression Across Distinct Ethnic Populations. Clin Cancer Res. 10: 5508-5517, 2004.

6. Ihmels J, Friedlander G, Bergmann S, Sarig O, Ziv Y, Barkai N. Revealing modular organization in the yeast transcriptional network. Nat Genet. 31(4): 370-7, 2002.

7. Ihmels J, Bergmann S, Barkai N. Defining transcription modules using large-scale gene expression data. Bioinformatics. Mar. 25, 2004

8. Yu K, Lee C H, Tan P H, Hong G S, Wee S B, Wong C Y, Tan P. A molecular signature of the Nottingham prognostic index in breast cancer. Cancer Res. 64(9): 2962-8, 2004.

9. Armes J E, Hammet F, De Silva M, Ciciulla J, Ramus S J, Soo W K, Mahoney A, Yarovaya N, Henderson M A, Gish K, Hutchins A M, Price G R, Venter D J. Candidate tumor-suppressor genes on chromosome arm 8p in early-onset and high-grade breast cancers. Oncogene. 23(33): 5697-702, 2004.

10. van't Veer, L. J., H. Dai, M. J. van de Vijver, Y. D. He, A. A. M. Hart, M. Mao, H. L. Peterse, K. van der Kooy, M. J. Marton, A. T. Witteveen, G. J. Schreiber, R. M. Kerkhoven, C. Roberts, P. S. Linsley, R. Bernards, and S. H. Friend. Gene expression profiling predicts clinical outcome of breast cancer. Nature 415, 530-536, 2002.

11. Lankat-Buttgereit B, Goke R. Programmed cell death protein 4 (pdcd4): a novel target for antineoplastic therapy? Biol Cell. 95(8): 515-9, 2003.

12. Nakayama K, Hatakeyama S, Maruyama S, Kikuchi A, Onoe K, Good R A, Nakayama K I. Impaired degradation of inhibitory subunit of NF-kappa B (I kappa B) and beta-catenin as a result of targeted disruption of the beta-TrCP1 gene. Proc Natl Acad Sci U S A. 100(15): 8752-7, 2003.

13. Cayli S, Sakkas D, Vigue L, Demir R, Huszar G. Cellular maturity and apoptosis in human sperm: creatine kinase, caspase-3 and Bcl-XL levels in mature and diminished maturity sperm. Mol Hum Reprod. 10(5): 365-72, 2004.

14. Jansen A P, Camalier C E, Stark C, Colburn N H. Characterization of programmed cell death 4 in multiple human cancers reveals a novel enhancer of drug sensitivity. Mol Cancer Ther. 3(2): 103-10, 2004.

15. Bieche I, Girault I, Urbain E, Tozlu S, Lidereau R. Relationship between intratumoral expression of genes coding for xenobiotic-metabolizing enzymes and benefit from adjuvant tamoxifen in estrogen receptor alpha-positive postmenopausal breast carcinoma. Breast Cancer Res. 6(3): R252-63, 2004.

16. Chang J C, Wooten E C, Tsimelzon A, Hilsenbeck S G, Gutierrez M C, Elledge R, Mohsin S, Osborne C K, Chamness G C, Allred D C, O'Connell P. Gene expression profiling for the prediction of therapeutic response to docetaxel in patients with breast cancer. Lancet. 362(9381): 362-9, 2003 58.

17. Naschberger E, Werner T, Vicente A B, Guenzi E, Topolt K, Leubert R, Lubeseder-Martellato C, Nelson P J, Sturzl M. Nuclear factor-kappaB motif and interferon-alpha-stimulated response element co-operate in the activation of guanylate-binding protein-1 expression by inflammatory cytokines in endothelial cells. Biochem J. 379(Pt 2): 409-20, 2004.

18. Espert L, Rey C, Gonzalez L, Degols G, Chelbi-Alix M K, Mechti N, Gongora C. The exonuclease ISG20 is directly induced by synthetic dsRNA via NF-kappaB and IRF1 activation. Oncogene. 23(26): 4636-40, 2004.

19. Pahl H L. Activators and target genes of Rel/NF-B transcription factors. Oncogene. 18: 6853-6866, 1999.

20. Biswas D K, Shi Q, Baily S, Strickland I, Ghosh S, Pardee A B, Iglehart J D. NF-kappa B activation in human breast cancer specimens and its role in cell proliferation and apoptosis. Proc Natl Acad Sci U S A. 101(27): 10137-42,

21. Karin M, Cao Y, Greten F R, Li Z W. NF-kappaB in cancer: from innocent bystander to major culprit. Nat Rev Cancer: 2(4): 301-10, 2002.

1. Method for predicting response to treatment in a patient with breast cancer, the method comprising assigning a prediction to the patient based on the expression levels of a set of genes in a breast tumour sample from said patient, wherein said set of genes comprises at least 10 genes selected from Table 2.

2. A method according to claim 1 wherein the set of genes comprises at least 20, 25, 30 or all of the genes of Table 2.

3. A method according to claim 1 wherein the set of genes comprise at least the first 5 genes listed in Table 2a.

4. (canceled)

5. (canceled)

6. (canceled)

7. A method according to claim 1 comprising the steps of providing an expression profile that represents the expression levels in the tumour of said set of genes; and assigning a prediction and/or treatment regimen to the patient based on the expression profile.

8. A method for determining the prognosis and/or treatment regimen of a patient with breast cancer said method comprising the steps of
(a) measuring the expression levels in a breast tumour sample obtained from said patient of a set of genes comprising at least 10 genes selected from Table 2;
(b) providing an expression profile that represents the expression levels in the tumour of said set of genes; and
(c) assigning a prognosis and/or treatment regimen to the patient based on the expression profile.

9. A method according to claim 8 wherein step (b) comprises contacting expression products obtained from the sample with binding members capable of binding to said expression products, said binding members being indicative of the expression of said set of genes, wherein such binding can be measured.

10. A method according to claim 9 wherein the expression products are selected from the group consisting of mRNA, cDNA, cRNA or expressed polypeptide.

11. A method according to claim 9 wherein the binding member is a complementary nucleic acid sequence or a specific antibody.

12. A method according to claim 9 wherein the expression products are labelled for detection.

13. A method according to claim 9 wherein the binding member is labelled for detection.

14. A method according to claim 8 wherein step (c) comprises comparing the expression profile from the breast tumour sample of the patient with previously obtained expression profiles and/or a previously determined standard profile which is characteristic of a particular prognosis and/or characteristic of a predictive response to treatment.

15. A method according to claim 14 wherein the previously obtained profiles are stored as a database of profiles.

16. A method according to claim 8 further comprising comparing the expression levels of the set of genes in the breast tumour sample before and after treatment to detect any change in the expression profile indicative of an improved prognosis or worsened prognosis.

17. A method according to claim 8 wherein an expression profile of the breast tumour sample has already determined the tumour to be an ER+ tumour subgroup.

18. A method according to claim 1 wherein the treatment is hormonal therapy and/or chemotherapy.

19. An apparatus for predicting response to treatment of a breast tumour sample, which apparatus comprises a solid support to which are attached a plurality of binding members, each binding member being capable of specifically and independently binding to an expression product of one of a set of genes, wherein the set of genes comprises at least 10 genes from Table 2.

20. An apparatus according to claim 19 wherein the set of genes comprises at least 20, 25, 30 or all of the genes of Table 2.

21. An apparatus according to claim 19 wherein the set of genes comprise at least the first 5 genes listed in Table 2a.

22. An apparatus according to claim 19 wherein the solid support has attached thereto only binding members that are capable of specifically and independently binding to expression products of the genes identified in Table 2.

23. An apparatus according to according to claim 17 comprising a nucleic acid microarray wherein the binding members are nucleic acid sequences.

24. (canceled)

25. (canceled)

26. A kit for predicting response to treatment in a patient with breast cancer, said kit comprising a plurality of binding members capable of specifically binding to expression products of a set of genes and a detection reagent, wherein the set of genes comprises at least 10 genes selected from Table 2, said kit optionally comprising the apparatus of claim 19.

27. A kit according to claim 26 further comprises means for labelling said plurality of binding members.

28. A kit according to claim 26, wherein the set of genes comprises at least 20, 25, 30 or all of the genes of Table 2.

29. A kit according to any claim 26 wherein the set of genes comprise at least the first 5 genes listed in Table 2a.

30. A kit according to claim 26 further comprising a data analysis tool, wherein the data analysis tool is a computer program.

31. A kit according to claim 30 wherein the data analysis tool comprises an algorithm adapted to discriminate between the expression profiles of tumours with predicted responses.

32. A kit according to claim 26 comprising expression profiles from breast tumour samples with known responses to treatment and/or expression profiles characteristic of a particular response to treatment.

33. (canceled)

34. (canceled)

35. (canceled)

36. A method of producing a nucleic acid expression profile for a breast tumour sample comprising the steps of
(a) isolating expression products from said breast tumour sample;
(b) identifying the expression levels of a set of genes, said set of genes comprising at least 10 genes selected from Table 2; and
(c) producing from the expression levels an expression profile for said breast tumour sample.

37. A method according to claim 36 wherein the set of genes comprises at least 20, 25, 30 or all of the genes of Table 2.

38. A method according to claim 36 wherein the set of genes comprise at least the first 5 genes listed in Table 2a.

39. A method according to claim 36 comprising adding the expression profile to a gene expression profile database.

40. A method according to claim 36 further comprising comparing the expression profile with a second expression profile or a plurality of expression profiles characteristic of a particular response to treatment.

41. A method according to claim 40 further comprising the step of producing a standard expression profile representing the first and second and/or the plurality of expression profiles characteristic of a particular response to treatment.

42. A method according to claim 40, comprising the steps of:
(a) isolating expression products from a first breast tumour sample; contacting said expression products with a plurality of binding members capable of specifically and independently binding to expression products of the set of genes; and creating a first expression profile from the expression levels of the set of genes in the tumour sample;
(b) isolating expression products from a second breast tumour sample of known prognosis, contacting said expression products with a plurality of binding members capable of specifically and independently binding to expression products of the set of genes of step (a) so as to create a comparable second expression profile of a breast tumour sample; and
(c) comparing the first and second expression profiles to determine the treatment response of the first breast tumour sample.

43. An expression profile database comprising a plurality of gene expression profiles of breast tumour samples, wherein the gene expression profiles are derived from expression levels of a set of genes, wherein the set of genes comprises at least 10 genes selected from Table 2, which database is retrievably held on a data carrier.

44. An expression profile database according to claim 43 wherein the set of data comprises at least 20, 25, 30 or all of the genes of Table 2.

45. An expression profile database according to claim 43 wherein the set of genes comprise at least the first 5 genes listed in Table 2a.

46. An expression profile database according to claim 43 wherein the expression profiles are nucleic acid expression profiles.

47. A method of determining a molecular subtype of a tumour, said method comprising
(a) obtaining gene expression products from a plurality of tumour samples;
(b) dividing said tumour samples into groups on the basis of the amount of gene expression product for a plurality of pre-selected genes above a pre-defined threshold; and
(d) allocating a molecular subtype to said group of tumours based on the gene expression profile.

48. A method according to claim 47 further comprising sub-dividing said tumour samples by repeating step (b) using a higher pre-selected threshold.

49. A diagnostic tool comprising a plurality of binding members capable of specifically and independently binding to expression products of at least 10 genes selected from Table 2, said plurality of binding members being fixed to a solid support.

50. A diagnostic tool according to claim 49 wherein at least 20, 25, 30 or all of the genes are selected from Table 2.

51. A diagnostic tool according to claim 49 or claim 50 wherein the set of genes comprise at least the first 5 genes listed in Table 2a.

52. A diagnostic tool according to claims 49 wherein the binding members are nucleic acid sequences.
